(19) United States
(12) Patent Application Publication
(10) Pub. No.: US 2016/0192841 A1
(43) Pub. Date: Jul. 7, 2016

(54) BIOLOGICAL INFORMATION MEASURING MODULE AND BIOLOGICAL INFORMATION MEASURING APPARATUS

(71) Applicant: SEIKO EPSON CORPORATION, Tokyo (JP)

(72) Inventors: Akira INAGAKI, Matsumoto-shi (JP); Hironori HASEI, Azumino-shi (JP)

(21) Appl. No.: 14/987,585

(22) Filed: Jan. 4, 2016

(30) Foreign Application Priority Data
Jan. 5, 2015 (JP) 2015-000108

(51) Int. Cl. A61B 5/00

(52) U.S. Cl. CPC A61B 5/0059; A61B 5/681; A61B 2562/0238; A61B 2562/04

(57) ABSTRACT

A biological information measuring apparatus includes a sensor unit as a biological information measuring module including a light emitting unit that emits light to an object, and a light receiving unit that receives light from the object. An interval between the light emitting unit and the light receiving unit is equal to or greater than 0.8 mm and equal to or less than 1.0 mm.

### CROSS-REFERENCES TO RELATED APPLICATIONS

This application claims priority to Japanese Patent Application No. 2015-000108, filed Jan. 5, 2015, the entirety of which is hereby incorporated by reference.

### BACKGROUND

1. Technical Field

The present invention relates to a biological information measuring module, and a biological information measuring apparatus including the biological information measuring module.

2. Related Art

Hitherto, there have been known measuring apparatuses that are worn around body parts, such as a wrist, by a band or the like and measure biological information such as a wearer's pulse waves, and wristwatch type electronic apparatuses having a function of measuring the biological information. For example, JP-A-2000-254105 discloses an arm mounted measuring apparatus which is worn around the arm of a wearer (test subject) and is mounted with a biological information measuring module that measures biological information, such as pulse waves, using an optical pulse wave detection sensor.

Such apparatuses (measuring apparatus, electronic apparatus) optically measure the flow of blood under a skin surface and convert the measured blood flow into a signal to thereby obtain biological information such as pulse waves, and thus a configuration of a dimensional relationship between a light emitting unit and a light receiving unit becomes significantly important. In particular, a distance between the light receiving unit and the light emitting unit is important and has great effect on measurement (sensing) results.

In addition, when such apparatuses (measuring apparatus, electronic apparatus) are used for the purposes related to, for example, sports, portability and reductions in size and weight are significantly important viewpoints for preventing the worn apparatuses from affecting the performance of wearers (test subjects). In addition, for example, even when the apparatuses are used for medical and health purposes, consideration for avoiding imposing a burden to patients or test subjects is required, and thus portability and reductions in size and weight are significantly important viewpoints. In this manner, apparatuses that are worn around body parts, such as a wrist, to thereby obtain biological information are required to rigorously seek an improvement in portability and a reduction in size and weight.

However, in the arm mounted measuring apparatus disclosed in JP-A-2000-254105, there is no description regarding a distance between the light receiving unit and the light emitting unit, and a problem in the above-mentioned configuration of a dimensional relationship between the light emitting unit and light receiving unit is not mentioned.

### SUMMARY

An advantage of some aspects of the invention is to solve at least a part of the problems described above, and the invention can be implemented as the following forms or application examples.

### Application Example 1

A biological information measuring module according to this application example includes a light emitting unit that emits light to an object, and a light receiving unit that receives light which is reflected by the object. An interval between the light emitting unit and the light receiving unit is equal to or greater than 0.4 mm and equal to or less than 1.7 mm.

When the interval between the light emitting unit and the light receiving unit is smaller than 0.4 mm, light emitted from the light emitting unit is directly incident on the light receiving unit, or the influence of disturbance light is exerted because a space for providing a light shielding unit such as a light shielding wall which shields disturbance light cannot be secured, and thus measurement cannot be performed accurately.

According to this application example, the interval between the light emitting unit and the light receiving unit is equal to or greater than 0.4 mm, and thus it is possible to prevent light emitted from the light emitting unit from directly entering the light receiving unit and to secure a space for providing, for example, a light shielding wall. In addition, as the interval between the light emitting unit and the light receiving unit becomes larger, the size of a measuring apparatus becomes larger. On the other hand, the interval between the light emitting unit and the light receiving unit is set to be equal to or less than 1.7 mm, and thus it is possible to realize the small-sized biological information measuring module having excellent portability.

Meanwhile, the wording “interval between the light emitting unit and the light receiving unit” as used herein refers to a distance between an outer circumferential side of the light emitting unit on the side opposite to the light receiving unit and an outer circumferential side of the light receiving unit on the side opposite to the light emitting unit.

### Application Example 2

In the biological information measuring module according to the application example, it is preferable that an interval between the light emitting unit and the light receiving unit is equal to or greater than 0.6 mm and equal to or less than 1.3 mm.

According to this application example, the interval between the light emitting unit and the light receiving unit is equal to or greater than 0.6 mm, and thus it is possible to more reliably prevent light emitted from the light emitting unit from directly entering the light receiving unit. In addition, the interval between the light emitting unit and the light receiving unit is set to be smaller than 1.3 mm, and thus it is possible to make the biological information measuring module smaller and to increase portability.

### Application Example 3

In the biological information measuring module according to the application example, it is preferable that an interval between the light emitting unit and the light receiving unit is equal to or greater than 0.8 mm and equal to or less than 1.0 mm.

According to this application example, the interval between the light emitting unit and the light receiving unit is set to be equal to or greater than 0.8 mm and equal to or less than 1.0 mm, and thus it is possible to increase the intensity of light received by the light receiving unit while securing a space for providing a light shielding unit such as a light shielding wall which shields disturbance light, which allows the stability of measurement to be achieved. In addition, the light intensity is high, and thus it is possible to widen a range of a mounting position of each of the light emitting unit and the light receiving unit which are capable of allowing a deterioration in the light intensity due to a variation in the mounting position, or the like, to easily perform mounting in manufacture, and to improve productivity. In addition, it is possible to provide the biological information measuring module having a smaller size and excellent portability.

### Application Example 4

It is preferable that the biological information measuring module according to the application example further includes a substrate and the light emitting unit and the light receiving unit are supported by the substrate.

According to this application example, the light emitting unit and the light receiving unit can be easily mounted on the substrate. In addition, since the light emitting unit and the light receiving unit are supported on the substrate, a distance from the light emitting unit and the light receiving unit to a measurement object is reduced, and thus it is possible to reduce noise to be mixed and to improve measurement accuracy.

### Application Example 5

In the biological information measuring module according to the application example, it is preferable that a reflective functional layer that reflects light emitted from the light emitting unit is provided in at least a portion of a vicinity of the light emitting unit.

According to this application example, light emitted from a peripheral direction of the light emitting unit can be made to be reflected by a reflective functional layer and to be directed to an object. Thereby, it is possible to increase the intensity (light emission intensity) of light directed to the object and to stabilize the measurement accuracy of biological information.

### Application Example 6

In the biological information measuring module according to the application example, it is preferable that a light shielding unit is provided between the light emitting unit and the light receiving unit.

According to this application example, disturbance light or stray light of reflected light, or unnecessary light such as direct light from the light emitting unit can be blocked by a light shielding unit such as a light shielding wall, and thus it is possible to perform detection (measurement) more accurately.

### Application Example 7

In the biological information measuring module according to the application example, it is preferable that the light shielding unit is provided so as to include at least one of a resin or a metal.

According to this application example, the light shielding unit can be easily formed of an inexpensive material. In addition, the light shielding unit can be configured to have excellent intensity by including a metal.

### Application Example 8

In the biological information measuring module according to the application example, it is preferable that an optical filter film is provided in a light receiving region of the light receiving unit.

According to this application example, it is possible to provide the optical filter in a smaller region and to provide the small-sized biological information measuring module.

### Application Example 9

In the biological information measuring module according to the application example, it is preferable that a light condensing unit that condenses light emitted from the light emitting unit is provided between the light emitting unit and the object.

According to this application example, since light emitted from the light emitting unit can be condensed by the light condensing unit so that the object is irradiated with the light, the intensity of light is increased, and thus it is possible to perform measurement more accurately.

### Application Example 10

In the biological information measuring module according to the application example, it is preferable that a plurality of the light emitting units are provided.

According to this application example, the plurality of light emitting units are provided, and thus it is possible to secure light emission intensity more sufficiently. In addition, biological information is detected by detecting light beams from the plurality of light emitting units, and thus it is possible to configure the biological information measuring module having further improved measurement accuracy.

### Application Example 11

In the biological information measuring module according to the application example, it is preferable that the plurality of light emitting units include a first light emitting unit and a second light emitting unit and an interval between the first light emitting unit and the light receiving unit and an interval between the second light emitting unit and the light receiving unit are the same as each other.

According to this application example, the length of a light path between the first light emitting unit and the light receiving unit and the length of a light path between the second light emitting unit and the light receiving unit are set to be substantially the same as each other, and light beams emitted from the first light emitting unit and the second light emitting unit are incident on the light receiving unit at substantially the same time, and thus it is possible to improve an S/N ratio.

### Application Example 12

In the biological information measuring module according to the application example, it is preferable that the plurality of light emitting units include a first light emitting unit and a second light emitting unit and an interval between the first light emitting unit and the light receiving unit and an interval between the second light emitting unit and the light receiving unit are different from each other.

According to this application example, the length of a light path between the first light emitting unit and the light receiving unit and the length of a light path between the second light emitting unit and the light receiving unit are different from each other, and a timing at which light is incident on the light receiving unit from the first light emitting unit and a timing at which light is incident on the light receiving unit from the second light emitting unit are different from each other, and thus it is possible to acquire a larger amount of biological information.

### Application Example 13

In the biological information measuring module according to the application example, it is preferable that an interval between the first light emitting unit and the second light emitting unit is equal to or greater than 1.0 mm and equal to or less than 4.9 mm.

According to this application example, the intensity of light can be increased, and thus it is possible to perform measurement accurately, to reduce the size of the biological information measuring module from a compact arrangement, and to increase portability such as, for example, a wearing feeling of a biological information measuring apparatus by using this module.

### Application Example 14

A biological information measuring apparatus according to this application example includes the biological information measuring module according to any one of the above-mentioned application examples.

According to this application example, detection (measurement) can be performed more accurately, and the biological information measuring module having a small size and excellent portability is provided, and thus it is possible to stably detect biological information and to provide the biological information measuring apparatus having a small size and excellent portability.

### BRIEF DESCRIPTION OF THE DRAWINGS

The invention will be described with reference to the accompanying drawings, wherein like numbers reference like elements.

FIGS. 1A and 1B are perspective views illustrating the exterior of a biological information measuring apparatus according to a first embodiment.

FIG. 2 is a side view illustrating the exterior of the biological information measuring apparatus of the first embodiment.

FIG. 3 is a diagram illustrating the wearing of the biological information measuring apparatus and communication with a terminal device.

FIG. 4 is a functional block diagram of the biological information measuring apparatus.

FIGS. 5A and 5B illustrate a sensor unit as a biological information measuring module, FIG. 5A is a front cross-sectional view, and FIG. 5B is a plan view seen from line A-A.

FIG. 6 is a graph illustrating the suitability of an interval between a light emitting unit and a light receiving unit.

FIG. 7 is a plan view illustrating Modification Example 1 of the arrangement of a light emitting unit and a light receiving unit.

FIGS. 8A and 8B are plan views respectively illustrating Modification Example 2 and Modification Example 3 of the arrangement of a light emitting unit and a light receiving unit.

FIGS. 9A and 9B are plan views respectively illustrating Modification Example 4 and Modification Example 5 of the arrangement of a light emitting unit and a light receiving unit.

FIG. 10 is a cross-sectional view illustrating an example of the related art of a biological information measuring apparatus according to a second embodiment.

FIG. 11 is a perspective view illustrating the biological information measuring apparatus according to the second embodiment.

FIG. 12 is a front view illustrating a biological information measuring apparatus according to a third embodiment.

FIG. 13 is a perspective view illustrating a biological information measuring apparatus according to a fourth embodiment.

FIG. 14 is a cross-sectional view illustrating a biological information measuring apparatus according to a fifth embodiment.

FIG. 15 is a flow chart illustrating a method of manufacturing the biological information measuring apparatus according to the second to fifth embodiments.

FIG. 16 is a schematic diagram illustrating a web page serving as a starting point of a health manager in a biological information measuring apparatus according to a sixth embodiment.

FIG. 17 is a diagram illustrating an example of a nutrition web page.

FIG. 18 is a diagram illustrating an example of an activity level web page.

FIG. 19 is a diagram illustrating an example of a mental concentration web page.

FIG. 20 is a diagram illustrating an example of a sleep web page.

FIG. 21 is a diagram illustrating an example of a daily activity web page.

FIG. 22 is a diagram illustrating an example of a health degree web page.

FIG. 23 is a partial cross-sectional view illustrating a modification example of a light receiving unit.

FIG. 24 is a partial cross-sectional view illustrating a modification example of a light emitting unit.

### DESCRIPTION OF EXEMPLARY EMBODIMENTS

Hereinafter, this embodiment will be described. Meanwhile, this embodiment described below does not improperly limit the contents of the invention which are described in the appended claims. In addition, all of the components described in this embodiment are not necessarily essential components of the invention.

### First Embodiment

### 1. Overall Configuration Example of Biological Information Measuring Apparatus

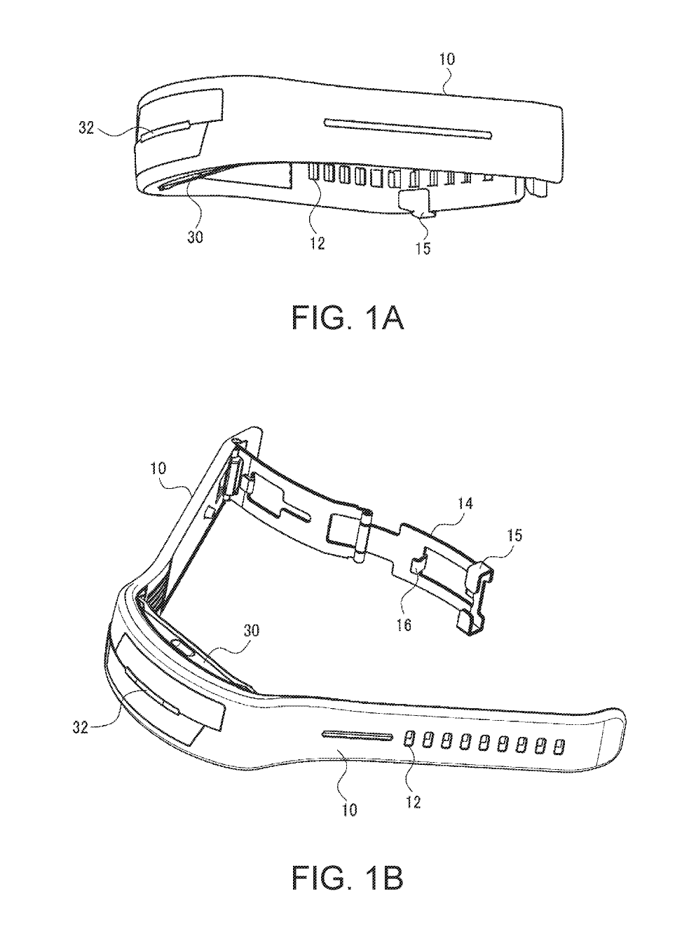

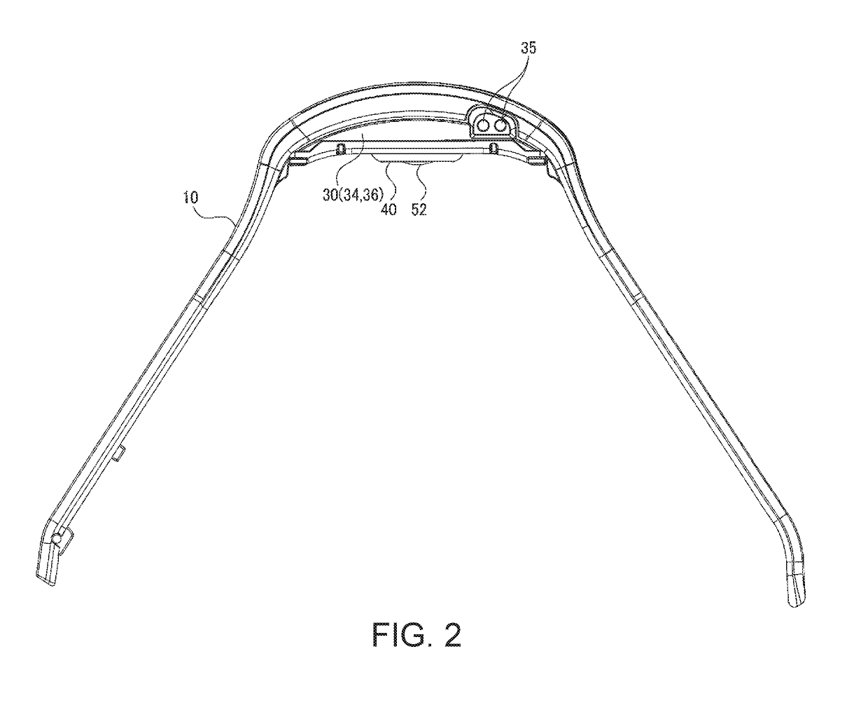

FIGS. 1A and 1B and FIG. 2 are schematic diagrams illustrating the exterior of a biological information measuring apparatus (biological information detecting apparatus) according to a first embodiment. FIG. 1A is a diagram when the biological information measuring apparatus is seen from the front, FIG. 1B is a diagram when the biological information measuring apparatus of FIG. 1A is obliquely seen from above, and FIG. 2 is a diagram when the biological information measuring apparatus is seen from the side.

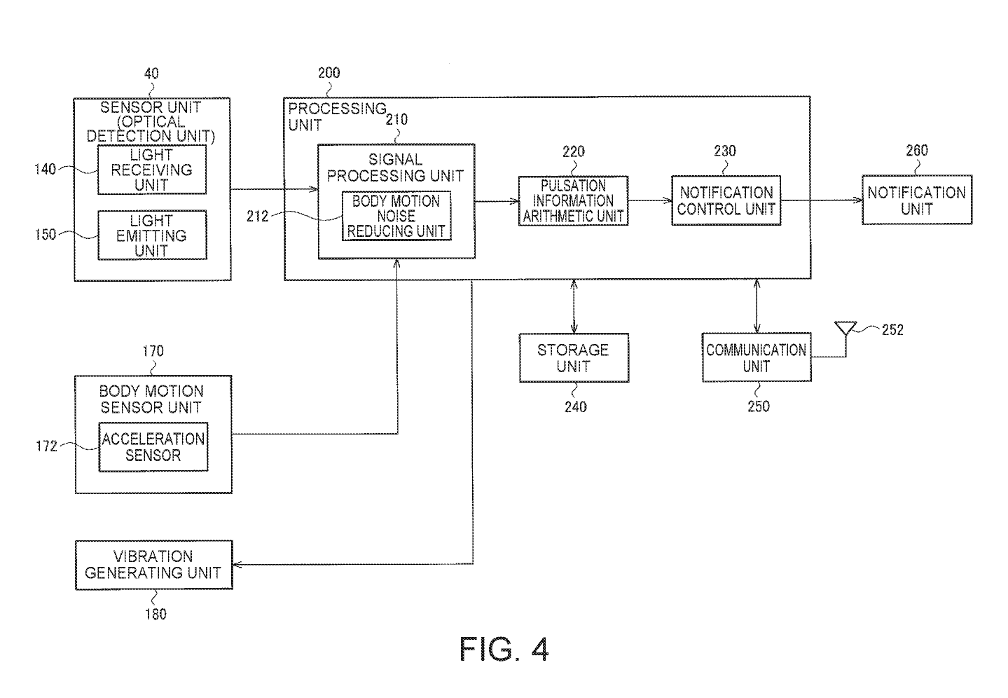

As illustrated in FIGS. 1A and 1B and FIG. 2, the biological information measuring apparatus of this embodiment includes a band portion 10, a case portion 30, and a sensor unit 40 as a biological information measuring module. The case portion 30 is attached to the band portion 10. The sensor unit 40 is provided in the case portion 30. In addition, the biological information measuring apparatus includes a processing unit 200 as illustrated in FIG. 4 to be described later. The processing unit 200 is provided in the case portion 30, and detects biological information on the basis of a detection signal from the sensor unit 40. Meanwhile, the biological information measuring apparatus of this embodiment is not limited to the configurations illustrated in FIG. 1A and FIG. 1B and FIG. 2, and various modifications such as the omission of some of the components thereof, replacement with other components, or the addition of other components can be made.

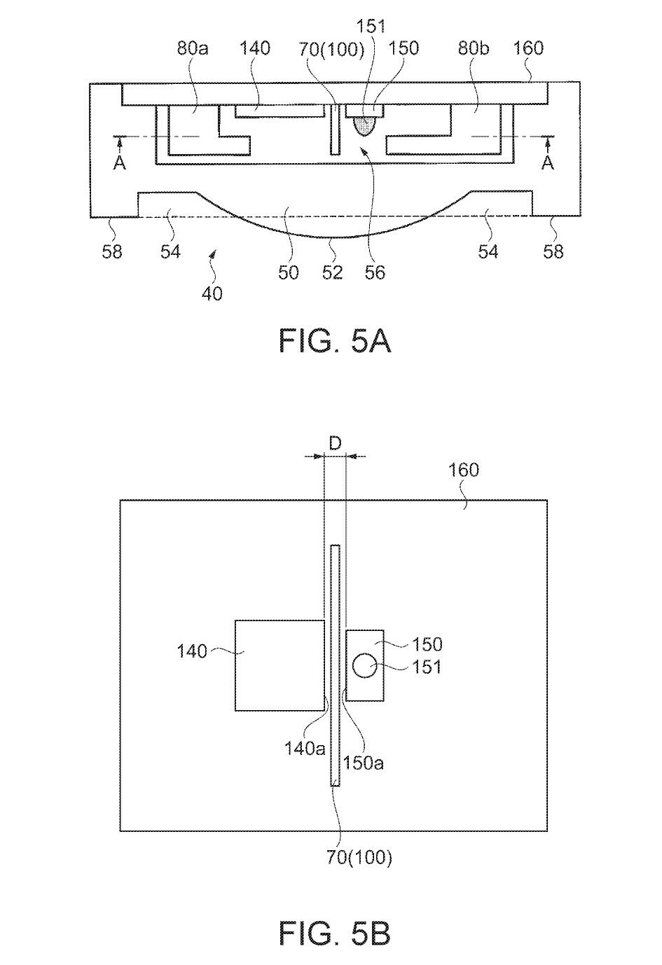

The sensor unit 40 as a biological information measuring module includes a substrate 160, a light emitting unit 150, a light receiving unit 140, a light shielding member 70, a light detection unit including a throttle portion 80 (80a, 80b), and other members, as described later with reference to FIGS. 5A and 5B. In the example illustrated in FIGS. 5A and 5B, the other members include a convex portion 52, a groove portion 54, a concave portion 56, a pressing suppressing portion 58, and the like which are realized by the light transmitting member 50. Here, a modification can also be made in which the light detection unit according to this embodiment includes the members, that is, the entire sensor unit 40 corresponds to the light detection unit.

Referring back to FIGS. 1A and 1B and FIG. 2, the band portion 10 is wound around the wrist of a wearer (hereinafter, also referred to as a user) so that the biological information measuring apparatus is worn thereon. The band portion 10 includes band holes 12 and a buckle portion 14. The buckle portion 14 includes a band insertion portion 15 and a protrusion portion 16. The user inserts one end side of the band portion 10 into the band insertion portion 15 of the buckle portion 14 and inserts the protrusion portion 16 of the buckle portion 14 into the band hole 12 of the band portion 10 to thereby wear the biological information measuring apparatus around his or her wrist. In this case, the magnitude of pressing (pressing against the surface of the wrist) by the sensor unit 40 to be described later is adjusted according to into which of the band holes 12 the protrusion portion 16 is inserted.

The case portion 30 is equivalent to a main body portion of the biological information measuring apparatus. Various components of the biological information measuring apparatus such as the sensor unit 40 and the processing unit 200 (see FIG. 4) are provided within the case portion 30. That is, the case portion 30 is a housing that accommodates the components. The case portion 30 includes, for example, a top case 34 which is positioned on the opposite side to the wrist and a bottom case 36 which is positioned on the wrist side. Meanwhile, the case portion 30 may not be configured so as to separate into the top case 34 and the bottom case 36.

The case portion 30 is provided with a light emitting window portion 32. The light emitting window portion 32 is formed of a light transmitting member. In addition, the case portion 30 is provided with a light emitting unit (LED, a light emitting unit for a notice which is different from the light emitting unit 150 of the light detection unit) which is mounted on a flexible substrate, and light from the light emitting unit is emitted to the outside of the case portion 30 through the light emitting window portion 32.

As illustrated in FIG. 2, the case portion 30 is provided with a terminal portion 35. When the biological information measuring apparatus is mounted on a cradle not shown in the drawing, a terminal portion of the cradle and the terminal portion 35 of the case portion 30 are electrically connected to each other. Thereby, a secondary battery (battery) provided in the case portion 30 can be charged.

The sensor unit 40 as a biological information measuring module detects biological information such as, for example, pulse waves of a test subject. For example, the sensor unit 40 includes a light receiving unit 140 and a light emitting unit 150 as illustrated in FIG. 4 and FIGS. 5A and 5B to be described later. In addition, the sensor unit 40 is formed of the light transmitting member 50 and includes the convex portion 52 that comes into contact with a test subject's skin surface and applies pressure. In this manner, the light emitting unit 150 emits light in a state where the convex portion 52 applies pressure to the skin surface, the light receiving unit 140 receives the light reflected by the test subject (blood vessel), and the light reception result thereof is output to the processing unit 200 as a detection signal. In addition, the processing unit 200 detects biological information, such as pulse waves, on the basis of the detection signal from the sensor unit 40. Meanwhile, biological information to be detected by the biological information measuring apparatus of this embodiment is not limited to pulse waves (pulse rate), and the biological information measuring apparatus may be an apparatus that detects biological information (for example, oxygen saturation in the blood, body temperature, heartbeat, and the like) other than pulse waves.

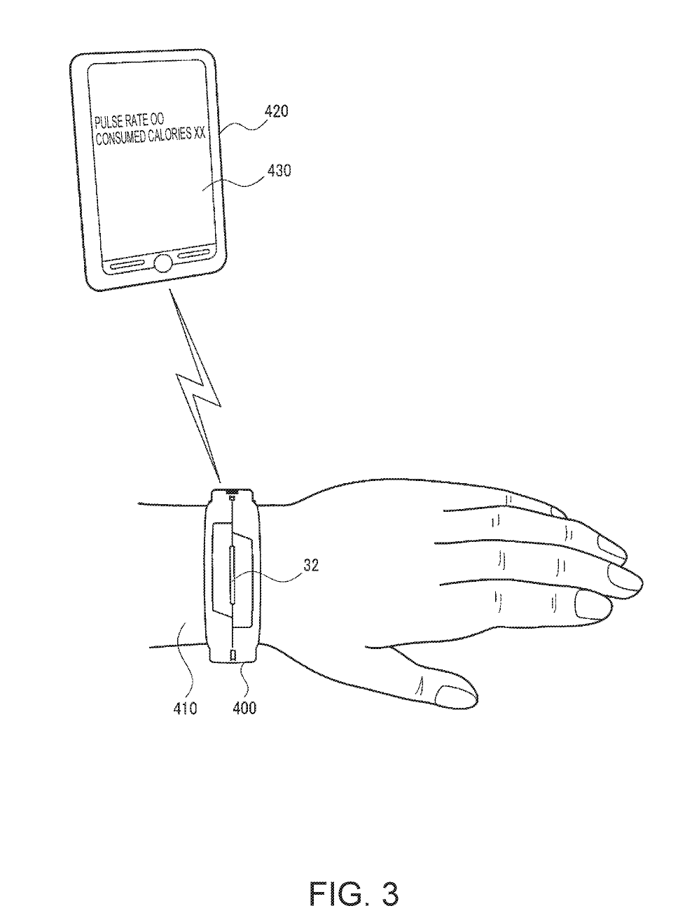

FIG. 3 is a schematic diagram illustrating the wearing of a biological information measuring apparatus 400 and communication with a terminal device 420. As illustrated in FIG. 3, a user who is a test subject wears the biological information measuring apparatus 400 around a wrist 410 like a wristwatch. As illustrated in FIG. 2, the sensor unit 40 is provided on a surface of the case portion 30 on the test subject side. Accordingly, when the biological information measuring apparatus 400 is worn, the convex portion 52 of the sensor unit 40 comes into contact with the skin surface of the wrist 410 and applies pressure. In this state, the light emitting unit 150 of the sensor unit 40 emits light, and the light receiving unit 140 receives the reflected light, and thus biological information such as pulse waves is detected.

The biological information measuring apparatus 400 and the terminal device 420 are connected to each other for communication, and thus data can be exchanged therebetween. The terminal device 420 is a portable communication terminal such as, for example, a smartphone, a mobile phone, or a feature phone. Alternatively, the terminal device 420 may be an information processing terminal such as a tablet computer. Proximity wireless communication such as, for example, Bluetooth (registered trademark) can be adopted as a communication connection between the biological information measuring apparatus 400 and the terminal device 420. In this manner, the biological information measuring apparatus 400 and the terminal device 420 are connected to each other for communication connection, and thus various pieces of information such as a pulse rate and consumed calories can be displayed on a display unit 430 (LCD or the like) of the terminal device 420. That is, various pieces of information obtained on the basis of the detection signal of the sensor unit 40 can be displayed. Meanwhile, the arithmetic processing of information such as a pulse rate or consumed calories may be performed by the biological information measuring apparatus 400, or at least a portion thereof may be performed by the terminal device 420.

The biological information measuring apparatus 400 is provided with the light emitting window portion 32, so that a user is notified of various pieces of information by light emission (lighting, blinking) of a light emitting body for a notice (not shown). For example, in the case of entering a fat combustion zone in information such as consumed calories or in the case of leaving the fat combustion zone, this is given notice of by the light emission of the light emitting body through the light emitting window portion 32. In addition, when an e-mail is received in the terminal device 420, the biological information measuring apparatus 400 is notified of the received e-mail from the terminal device 420. The light emitting body of the biological information measuring apparatus 400 emits light, and thus a user is notified of the reception of an e-mail or the like.

In this manner, in the example illustrated in FIG. 3, the biological information measuring apparatus 400 is not provided with a display unit such as an LCD, and thus information required to be given notice of by characters or numerals is displayed on the display unit 430 of the terminal device 420. In this manner, in the example illustrated in FIG. 3, a user is notified of the necessary minimum information by the light emission of the light emitting body without providing a display unit such as an LCD, thereby realizing a reduction in the size of the biological information measuring apparatus 400. In addition, the biological information measuring apparatus 400 is not provided with a display unit, and thus it is possible to improve the beauty of the biological information measuring apparatus 400.

FIG. 4 is a functional block diagram of the biological information measuring apparatus of this embodiment. The biological information measuring apparatus illustrated in FIG. 4 includes the sensor unit 40 as a biological information measuring module, a body motion sensor unit 170, a vibration generating unit 180, the processing unit 200, a storage unit 240, a communication unit 250, an antenna 252, and a notification unit 260. Meanwhile, the biological information measuring apparatus of this embodiment is not limited to the configuration illustrated in FIG. 4, and various modifications such as the omission of some of the components thereof, replacement with other components, or the addition of other components can be made.

The sensor unit 40 as a biological information measuring module detects biological information such as pulse waves, and includes the light receiving unit 140 and the light emitting unit 150. A pulse wave sensor (photoelectric sensor) is realized by the light receiving unit 140, the light emitting unit 150, and the like. The sensor unit 40 outputs a signal detected by the pulse wave sensor as a pulse wave detection signal.

The body motion sensor unit 170 outputs a body motion detection signal which is a signal varying in response to body motion, on the basis of pieces of sensor information of various sensors. The body motion sensor unit 170 includes, for example, an acceleration sensor 172 as a body motion sensor. Meanwhile, the body motion sensor unit 170 may include a pressure sensor, a gyro sensor, or the like as the body motion sensor.

The processing unit 200 performs various types of signal processes and control processes, for example, with the storage unit 240 as a work area, and can be realized by, for example, a processor such as a CPU or a logic circuit such as an ASIC. The processing unit 200 includes a signal processing unit 210, a pulsation information arithmetic unit 220, and a notification control unit 230.

The signal processing unit 210 performs various types of signal processes (filtering and the like), and performs signal processing on, for example, a pulse wave detection signal from the sensor unit 40, a body motion detection signal from the body motion sensor unit 170, or the like. For example, the signal processing unit 210 includes a body motion noise reducing unit 212. The body motion noise reducing unit 212 performs processing for reducing (removing) body motion noise which is noise caused by body motion, from the pulse wave detection signal, on the basis of the body motion detection signal from the body motion sensor unit 170. Specifically, the body motion noise reducing unit performs a noise reduction process using, for example, an adaptive filter.

The pulsation information arithmetic unit 220 performs arithmetic processing of pulsation information on the basis of a signal from the signal processing unit 210, and the like. The pulsation information is information such as, for example, a pulse rate. Specifically, the pulsation information arithmetic unit 220 obtains a spectrum by performing frequency analysis processing such as FFT on the pulse wave detection signal having been subjected to the noise reduction process by the body motion noise reducing unit 212, and performs a process of setting a representative frequency in the obtained spectrum as a frequency of a heartbeat. A value obtained by increasing the obtained frequency by 60 times is set to be a pulse rate (heart rate) which is generally used. Meanwhile, the pulsation information is not limited to the pulse rate itself, and may be various other pieces of information (for example, the frequency or cycle of a heartbeat) which indicate, for example, a pulse rate.

In addition, the pulsation information may be information indicating the state of pulsation, or a value indicating, for example, the amount of blood itself may be set as pulsation information.

The notification control unit 230 controls the notification unit 260. The notification unit 260 (notification device) notifies a user of various pieces of information under the control of the notification control unit 230. For example, a light emitting body for a notice can be used as the notification unit 260. In this case, the notification control unit 230 controls a current flowing to an LED to thereby control the lighting, blinking, or the like of the light emitting body. Meanwhile, the notification unit 260 may be a display unit, such as an LCD, a buzzer, or the like.

In addition, the notification control unit 230 controls the vibration generating unit 180. The vibration generating unit 180 notifies a user of various pieces of information by vibration. The vibration generating unit 180 can be realized by, for example, a vibration motor (vibrator). The vibration motor generates vibration, for example, by rotating an eccentric weight. Specifically, the eccentric weight is attached to both ends of a driving shaft (rotor shaft) so that the motor itself shakes. The vibration of the vibration generating unit 180 is controlled by the notification control unit 230. Meanwhile, the vibration generating unit 180 is not limited to such a vibration motor, and various modifications can be made. The vibration generating unit 180 may be realized by, for example, a piezo element.

For example, a notice of start-up at the time of power-on, a notice of the first success in detecting pulse waves, a warning when a pulse-wave undetectable state is continued for a fixed period of time, a notice at the time of the movement of a fat combustion zone, a warning at the time of a battery voltage drop, a notice of a wake-up alarm, or a notice of an e-mail or a call from a terminal device such as a smartphone can be performed by the vibration of the vibration generating unit 180. Meanwhile, the pieces of information may be given notice of by a light emitting unit for a notice, or may be given notice of by both the vibration generating unit 180 and the light emitting unit.

The communication unit 250 performs communication with the external terminal device 420 as described in FIG. 3. For example, the communication unit performs wireless communication according to a standard such as Bluetooth (registered trademark). Specifically, the communication unit 250 receives a signal from the antenna 252 and transmits a signal to the antenna 252. The function of the communication unit 250 can be realized by a processor for communication or a logic circuit such as an ASIC.

### 2. Configuration Example of Sensor Unit as Biological Information Measuring Module

A detailed configuration example of the sensor unit as a biological information measuring module will be described below with reference to FIGS. 5A and 5B. FIGS. 5A and 5B are diagrams illustrating a detailed configuration example of the sensor unit 40. FIG. 5A is a front cross-sectional view, and FIG. 5B is a plan view seen from line A-A. Meanwhile, in FIG. 5B, the arrangement of the light receiving unit 140, the light emitting unit 150, and the light shielding member 70 (light shielding wall 100) as a light shielding unit is shown, and other components are not shown.

The sensor unit 40 includes the light receiving unit 140 and the light emitting unit 150. The light receiving unit 140 and the light emitting unit 150 are mounted on the substrate 160 (sensor substrate) at a predetermined interval. The light emitting unit 150 emits light to an object (test subject or the like), and the light receiving unit 140 receives light (reflected light, transmitted light, or the like) from the object. For example, when the light emitting unit 150 emits light and the light is reflected by an object (for example, a blood vessel), the light receiving unit 140 receives the reflected light and detects. The light receiving unit 140 can be realized by a light receiving element such as, for example, a photodiode. The light emitting unit 150 can be realized by a light emitting element such as, for example, an LED. For example, the light receiving unit 140 can be realized by a diode element of a PN junction which is formed on a semiconductor substrate, and the like. In this case, an angle limiting filter for narrowing a light reception angle or a wavelength limiting filter (optical filter film) that limits a wavelength of light incident on a light receiving element may be formed on the diode element.

Meanwhile, a dome-type lens 151 (condensing lens in a broad sense), as a light condensing unit, which is provided in the light emitting unit 150 is a lens for condensing light from an LED chip (light emitting element chip in a broad sense) which is resin-sealed (sealed with a light transmitting resin) in the light emitting unit 150. That is, in the light emitting unit 150 which is a surface-mounted type, the LED chip is disposed below the dome-type lens 151, and light from the LED chip is condensed by the dome-type lens 151 and is emitted to an object. Thereby, the intensity of light with which the object is irradiated can be increased, and thus it is possible to improve optical efficiency and to perform accurate measurement.

When a pulsimeter is taken as an example of the biological information measuring apparatus, light from the light emitting unit 150 travels within a test subject which is an object, and is diffused or scattered to epidermis, dermis, subcutaneous tissue, and the like. Thereafter, the light reaches a blood vessel (part to be detected) and is reflected. At this time, a portion of the light is absorbed into the blood vessel. Since the absorption of the light at the blood vessel varies by the influence of pulses and the amount of reflected light also varies, the light receiving unit 140 receives the reflected light and detects variations in the amount of light, and thus it is possible to detect a pulse rate which is biological information, and the like.

Such a biological information measuring apparatus optically measures the blood flow under a skin surface and converts the blood flow into a signal to thereby obtain biological information such as pulses, and thus an interval (distance) D between the light emitting unit 150 and the light receiving unit 140 becomes a significantly important element for the accuracy and stability of measurement. For example, when the interval D between the light receiving unit 140 and the light emitting unit 150 is not set to be large to a certain degree, light emitted from the light emitting unit 150 is directly incident on the light receiving unit 140, or the influence of disturbance light is easily exerted because a space for providing the light shielding member 70 (light shielding wall 100) as a light shielding unit that shields disturbance light cannot be secured, which results in a deterioration in the accuracy of measurement (a deterioration in measurement accuracy). On the other hand, when the interval D between the light receiving unit 140 and the light emitting unit 150 is excessively increased, the biological information measuring apparatus becomes larger, which results in a problem of portability deterioration such as a burden to wearing a unit around an arm (wrist). Here, the interval D indicates a distance between an outer circumferential side 150a of the light emitting unit 150 on the side opposite to the light receiving unit 140 and an outer circumferential side 140a of the light receiving unit 140 on the side opposite to the light emitting unit.

Specifically, portability becomes a significantly important viewpoint from consideration for preventing the worn biological information measuring apparatus from affecting the performance of a wearer (test subject) when the apparatus is used for the purposes related to, for example, sport, or consideration for avoiding imposing a burden to a patient or a wearer (test subject) when the apparatus is used for medical and health purposes.

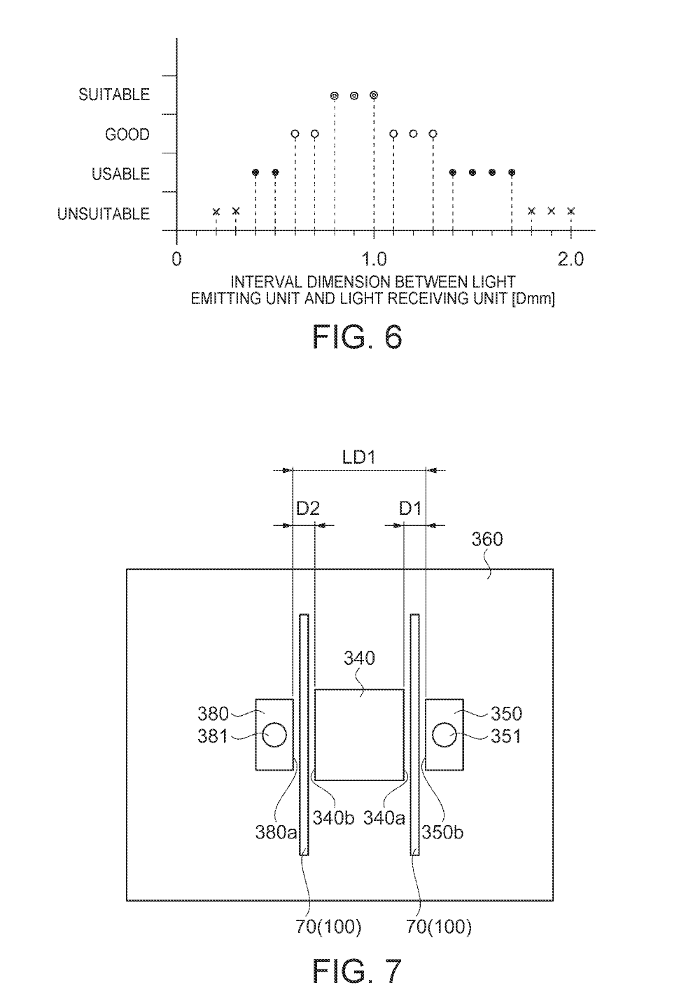

From such a viewpoint, the inventors have found a dimensional relationship which is excellent in portability while securing the accuracy and stability of measurement with respect to the interval D between the light emitting unit 150 and the light receiving unit 140 by wholeheartedly examining and verifying a configuration of a dimensional relationship between the light emitting unit 150 and the light receiving unit 140. Hereinafter, a preferable interval D between the light emitting unit 150 and the light receiving unit 140 will be described with reference to FIG. 6. Here, FIG. 6 is a graph illustrating verification results of suitability according to an interval between the light emitting unit and the light receiving unit. Meanwhile, in the graph illustrated in FIG. 6, a horizontal axis represents an interval D (interval D illustrated in FIG. 5B) which is an interval dimension between the light emitting unit 150 and the light receiving unit 140, and verification results (determination results) of suitability in the respective intervals D which are interval dimensions are shown.

### Interval Between Light Emitting Unit and Light Receiving Unit

Regarding an interval between the light emitting unit 150 and the light receiving unit 140, the intensity of a detection signal increases as the interval D becomes smaller, that is, as the light emitting unit 150 and the light receiving unit 140 become closer to each other, and thus detection performance such as sensitivity is improved. In addition, as the interval D between the light emitting unit 150 and the light receiving unit 140 becomes smaller, that is, as the light emitting unit 150 and the light receiving unit 140 become closer to each other, it is more suitable for a reduction in the size of the biological information measuring apparatus. However, when the interval D is excessively reduced, there is a tendency for light emitted from the light emitting unit 150 to directly enter the light receiving unit 140, which results in a deterioration in the accuracy of measurement (detection) in the light receiving unit 140 by the light. On the other hand, when the interval D is increased, it is possible to prevent light emitted from the light emitting unit 150 from directly entering the light receiving unit 140 and to prevent the accuracy of measurement (detection) of the light receiving unit 140 from deteriorating. However, when the interval D is excessively increased, it is necessary to increase the light emission intensity of the light emitting unit 150 in order to cope with the attenuation of light due to an increase in the length of a light path until light emitted from the light emitting unit 150 reaches the light receiving unit 140. Therefore, the consumption of power for light emission is increased, which leads to a concern for the occurrence of performance deterioration such as a reduction in an operating time when carried. In addition, when the interval D is excessively increased, the size of the sensor unit 40 is increased, which is not suitable for a reduction in the size of the biological information measuring apparatus.

Specifically, the interval D being equal to or less than 0.3 mm can make the size of the sensor unit 40 small and is suitable for a reduction in the size of the biological information measuring apparatus, but there is a tendency for light (scattered light) emitted from the light emitting unit 150 to directly enter the light receiving unit 140. In addition, when the interval D is equal to or less than 0.3 mm, it becomes difficult to insert the light shielding member 70 (light shielding wall 100) to be described later. Accordingly, a larger amount of noise is generated, and thus it is also difficult to create an algorithm for removing noise. Therefore, the accuracy of measurement (detection) in the light receiving unit 140 deteriorates. On the other hand, when the interval D is set to be equal to or greater than 1.8 mm, the size of the sensor unit 40 becomes larger. Consequently, the biological information measuring apparatus becomes larger, and thus there is a concern of a disadvantage, such as an uncomfortable feeling during the wearing thereof. In addition, since a path until light emitted from the light emitting unit 150 reaches an object, and reflected light reaches the light receiving unit 140 increases, there is a concern of noise having a tendency to be added. Accordingly, as illustrated in FIG. 6, when the interval D between the light emitting unit 150 and the light receiving unit 140 is equal to or less than 0.3 mm and equal to or greater than 1.8 mm, it is determined that the units are not suitable (unsuitable) for use. In other words, when the interval D between the light emitting unit 150 and the light receiving unit 140 is within a range from equal to or greater than 0.4 mm to equal to or less than 1.7 mm, the units can be suitably used for the biological information measuring apparatus. A detailed description thereof will be given below.

More specifically, when the interval D is set to be equal to or greater than 0.4 mm, scattered light directly received by the light receiving unit 140 is slightly generated, but it is possible to confirm that a detection result can be obtained to such a degree that the result becomes a standard as biological information. In addition, it is possible to insert the light shielding member 70 (light shielding wall 100) and to suppress scattered light.

In addition, when the interval D is equal to or less than 1.7 mm, it is possible to confirm that measurement accuracy does not deteriorate even when the amount of power for light emission in the light emitting unit 150 is not increased. Further, the sensor unit 40 mounted with the light emitting unit 150 also has an allowable size. Consequently, the biological information measuring apparatus can be configured as a small-sized apparatus.

Further, when the interval D is set to be equal to or greater than 0.6 mm, scattered light directly received by the light receiving unit 140 is slightly generated, but it is possible to confirm that a detection result can be obtained to such a degree that the result becomes a standard as biological information.

In addition, when the interval D is equal to or less than 1.3 mm, the element size of the light emitting unit 150 is significantly reduced, and thus it is possible to further reduce the size of the sensor unit 40 and to realize the small-sized biological information measuring apparatus.

In this manner, in the sensor unit 40, the interval D between the light emitting unit 150 and the light receiving unit 140 is set to be within a range from equal to or greater than 0.6 mm and equal to or less than 1.3 mm, and thus the units can be further suitably used for the biological information measuring apparatus.

Further, when the interval D is set to be equal to or greater than 0.8 mm, scattered light directly received by the light receiving unit 140 mostly disappears, and thus it is possible to confirm that biological information is detected (measured) with a high degree of accuracy.

In addition, the interval D is set to be equal to or less than 1.0 mm, and thus it is possible to reduce the size of the sensor unit 40 and to improve the arbitrariness of the arrangement of components constituting the sensor unit 40. Accordingly, it is possible to contribute to a more compact design of the biological information measuring apparatus and to realize the small-sized biological information measuring apparatus. Thereby, it is possible to configure the biological information measuring apparatus of which the wearing can be maintained without causing an uncomfortable feeling even when, for example, an unexpected impact is applied thereto.

In this manner, in the sensor unit 40, the interval D between the light emitting unit 150 and the light receiving unit 140 is set to be within a range from equal to or greater than 0.8 mm and equal to or less than 1.0 mm, and thus the units can be particularly suitably used for the biological information measuring apparatus.

A description will be given by referring back to FIGS. 5A and 5B. The light shielding member 70 (light shielding wall 100) as a light shielding unit is provided between the light receiving unit 140 and the light emitting unit 150. The light shielding member 70 (light shielding wall 100) prevents light from, for example, the light emitting unit 150 (direct light or the like) from being directly incident on the light receiving unit 140. The light shielding member 70 (light shielding wall 100) can be formed by, for example, sheet metal working of a metal plate. In this manner, when the light shielding member 70 (light shielding wall 100) is formed by sheet metal working of a metal plate, the light shielding member 70 (light shielding wall 100) having excellent strength can be easily formed of an inexpensive material. Meanwhile, an example of a material of the light shielding member 70 (light shielding wall 100) includes a resin such as rubber (including a natural resin and a synthetic resin) as a material other than a metal material. These materials can be easily obtained at a low cost, and allows the light shielding member 70 (light shielding wall 100) to be easily formed.

The light shielding member 70 as a light shielding unit is a member for shielding light. In this embodiment, the light shielding member 70 is provided between the light receiving unit 140 and the light emitting unit 150 as the light shielding wall 100, and shields the light receiving unit 140. Meanwhile, the light shielding member 70 may be provided so as to cover a portion of the light receiving unit 140, and may be configured to shield light incident on the light receiving unit 140. It is possible to improve detection performance while preventing light from the light emitting unit 150 from being incident on the light receiving unit 140, by the light shielding member 70 (light shielding wall 100).

In addition, it is preferable to perform a reflection suppressing process on at least the surface of the light receiving unit 140 on the side of the light shielding member 70 (light shielding wall 100) as a light shielding unit. For example, the light shielding member 70 is configured to have a surface (inner surface or the like) having a predetermined color such as a black color so that the irregular reflection of light is prevented. Alternatively, the light shielding member 70 may be configured to have a surface having a moth-eye structure. For example, a concavo-convex structure having several tens to several hundreds of cycles is formed in the surface of the light shielding member so as to configure a reflection preventing structure. When such a reflection suppressing process is performed, it is possible to effectively suppress the occurrence of a situation in which, for example, reflected light on the surface of the light shielding member 70 changes to stray light and becomes a noise component of a detection signal.

The light receiving unit 140, the light emitting unit 150, and the light shielding member 70 (light shielding wall 100) as a light shielding unit are mounted on the substrate 160. The substrate 160 is a, for example, rigid substrate. The substrate 160 is provided with a terminal (not shown) for connection to a terminal (not shown) of a signal and a power supply of the light receiving unit 140 and a terminal (not shown) for connection to a signal and a power supply of an external main substrate. For example, the terminal of the light receiving unit 140 and the terminal of the substrate 160 are connected to each other by wire bonding or the like.

In this manner, the light receiving unit 140, the light emitting unit 150, the light shielding member 70 (light shielding wall 100) as a light shielding unit, and the like are mounted (supported) on the substrate 160, and thus a distance from the light emitting unit 150 and the light receiving unit 140 to a measurement object is reduced. Thereby, it is possible to reduce noise mixed in light and to improve measurement accuracy.

In addition, the sensor unit 40 is provided with the throttle portions 80a and 80b. The throttle portion 80 narrows light from a test subject in a light path between the test subject and the sensor unit 40, and narrows light from the light emitting unit 150. In FIGS. 5A and 5B, the throttle portions 80a and 80b are provided between the light transmitting member 50 and the light emitting unit 150. Here, the throttle portions 80a and 80b may be provided between the light transmitting member 50 and a test subject or within the light transmitting member 50.

The light transmitting member 50 is provided on a surface of the biological information measuring apparatus which comes into contact with a test subject, and transmits light from the test subject. In addition, the light transmitting member 50 comes into contact with the test subject when biological information of the test subject is measured. For example, the convex portion 52 (detection window) of the light transmitting member 50 comes into contact with the test subject. Meanwhile, it is preferable that the shape of the surface of the convex portion 52 is a curved surface shape (spherical shape). However, the invention is not limited thereto, and various shapes can be adopted. In addition, the light transmitting member 50 may be a member capable of transmitting a wavelength of light from a test subject, and a transparent material or a colored material may be used.

The groove portion 54 for suppressing a pressing fluctuation or the like is provided in the vicinity of the convex portion 52 of the light transmitting member 50. In addition, when a surface of the light transmitting member 50 which is provided with the convex portion 52 is set to be a first surface, the light transmitting member 50 has the concave portion 56 at a position corresponding to the convex portion 52 in a second surface on the back side of the first surface. The light receiving unit 140, the light emitting unit 150, the light shielding member 70, and the throttle portions 80a and 80b are provided in a space of the concave portion 56.

In addition, the pressing suppressing portion 58 that suppresses pressing applied to a test subject (skin of a wrist) by the convex portion 52 is provided on a surface of the biological information measuring apparatus on a test subject side. In FIGS. 5A and 5B, the pressing suppressing portion 58 is provided so as to surround the convex portion 52 of the light transmitting member 50. The convex portion 52 protrudes toward the test subject side further than a pressing suppressing portion (pressing suppressing surface) 58.

It is possible to apply initial pressing for exceeding, for example, a vein vanishing point to a test subject by providing the convex portion 52. In addition, the pressing suppressing portion 58 for suppressing pressing applied to the test subject by the convex portion 52 is provided, and thus it is possible to minimally suppress a pressing fluctuation in a usage range in which the measurement of biological information is performed by the biological information measuring apparatus and to achieve a reduction in a noise component and the like. In addition, when the convex portion 52 protrudes from the pressing suppressing portion 58, the convex portion 52 comes into contact with the test subject and applies initial pressing, and then the pressing suppressing portion 58 comes into contact with the test subject, and thus it is possible to suppress pressing applied to the test subject by the convex portion 52. The wording “vein vanishing point” as used herein refers to a point in which a signal caused by a vein superimposed on a pulse wave signal vanishes or becomes smaller to the extent that the signal does not affect the measurement of pulse waves, when the convex portion 52 is brought into contact with the test subject and the strength of pressing is sequentially increased.

According to the above-mentioned configuration of the first embodiment, the interval D between the light emitting unit 150 and the light receiving unit 140 is accurately set, and thus it is possible to maintain and improve light emission intensity and light reception sensitivity and to provide the biological information measuring apparatus having a small size and excellent portability while securing the accuracy and stability of measurement.

### Modification Example of Arrangement of Light Emitting Unit and Light Receiving Unit

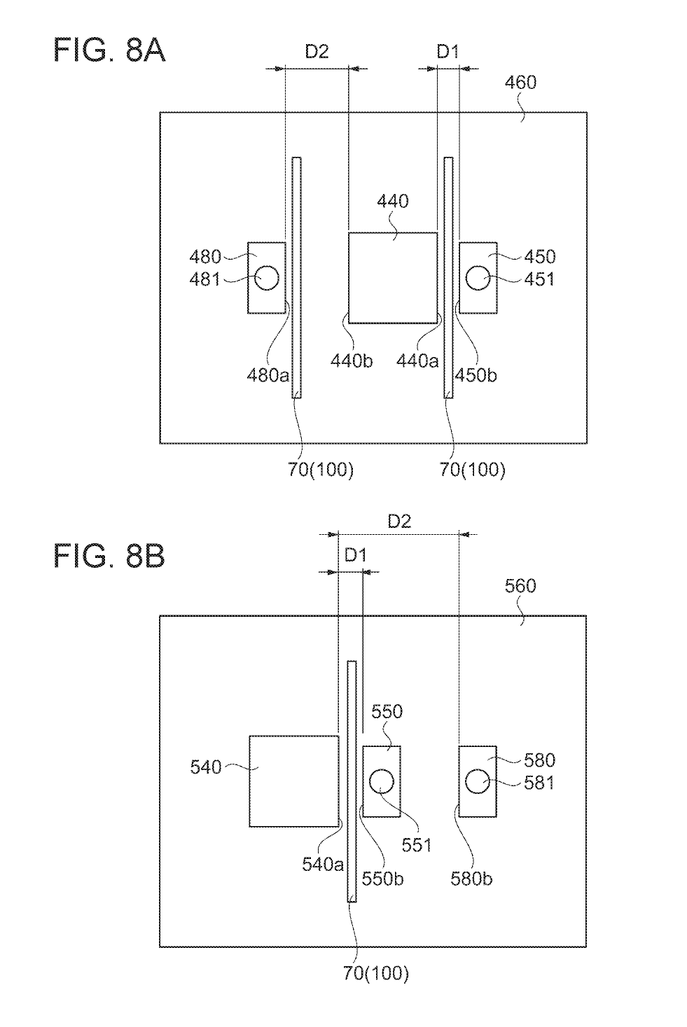

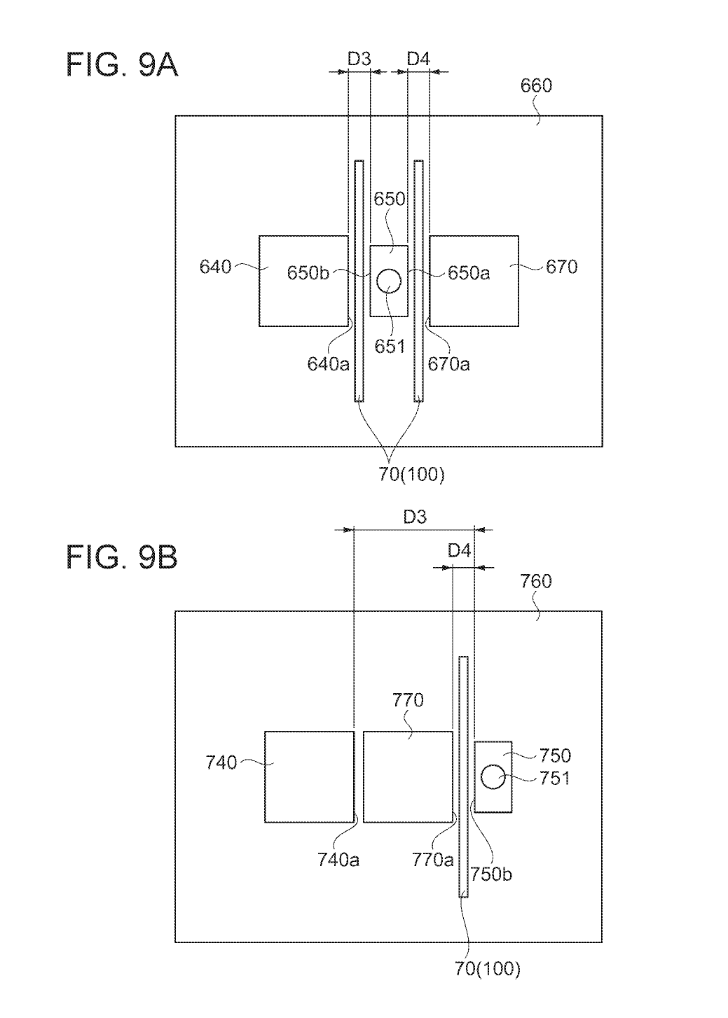

Next, a modification example of the arrangement of a light emitting unit and a light receiving unit will be described with reference to FIG. 7, FIGS. 8A and 8B, and FIGS. 9A and 9B. FIG. 7 is a plan view illustrating Modification Example 1 of the arrangement of a light emitting unit and a light receiving unit. In addition, FIGS. 8A and 8B illustrate a modification example of the arrangement of a light emitting unit and a light receiving unit. FIG. 8A is a plan view illustrating Modification Example 2, and FIG. 8B is a plan view illustrating Modification Example 3. FIGS. 9A and 9B illustrate a modification example of the arrangement of a light emitting unit and a light receiving unit. FIG. 9A is a plan view illustrating Modification Example 4, and FIG. 9B is a plan view illustrating Modification Example 5. Meanwhile, hereinafter, the same components as those in the above-described embodiment will be denoted by the same reference numerals and signs, and a description thereof may be omitted or simplified.

### Modification Example 1 of Arrangement

First, Modification Example 1 of the arrangement of a light emitting unit and a light receiving unit will be described with reference to FIG. 7. In the first embodiment described above, one light emitting unit 150 and one light receiving unit 140 are mounted on the substrate 160 (sensor substrate) so as to be lined up. In a configuration of Modification Example 1, a plurality of light emitting units and one light receiving unit are provided. A first light emitting unit 350 and a second light emitting unit 380 as a plurality of light emitting units, and a light receiving unit 340 are mounted on a substrate 360 so as to be lined up in a row along a predetermined direction in the order of the first light emitting unit 350, the light receiving unit 340, and the second light emitting unit 380. The first light emitting unit 350, the second light emitting unit 380, and the light receiving unit 340 are disposed so that a first interval D1 which is an interval between the first light emitting unit 350 and the light receiving unit 340 and a second interval D2 which is an interval between the second light emitting unit 380 and the light receiving unit 340 are set to be substantially the same distance. In addition, a light shielding member 70 (light shielding wall 100) as a light shielding unit is provided between the first light emitting unit 350 and the light receiving unit 340 and between the second light emitting unit 380 and the light receiving unit 340.

Specifically, the first light emitting unit 350, the light receiving unit 340, and the second light emitting unit 380 are disposed so that a first interval D1 which is a distance between an outer circumferential side 350b of the first light emitting unit 350 on the light receiving unit 340 side and an outer circumferential side 340a of the light receiving unit 340 on the first light emitting unit 350 side and a second interval D2 which is a distance between an outer circumferential side 380a of the second light emitting unit 380 on the light receiving unit 340 side and an outer circumferential side 340b of the light receiving unit 340 on the second light emitting unit 380 side are set to be the same as each other.

According to Modification Example 1, the plurality of light emitting units (in this example, the first light emitting unit 350 and the second light emitting unit 380) are provided, and thus it is possible to sufficiently secure light emission intensity by light emitted from the plurality of light emitting units. In addition, biological information is detected by detecting light beams from the plurality of light emitting units, and thus it is possible to further improve measurement accuracy.

By this arrangement, the length of a light path between the first light emitting unit 350 and the light receiving unit 340 and the length of a light path between the second light emitting unit 380 and the light receiving unit 340 are set to be substantially the same as each other, and light beams emitted from the first light emitting unit 350 and the second light emitting unit 380 are incident on the light receiving unit 340 at substantially the same time, and thus it is possible to improve an S/N ratio. That is, it is possible to improve the measurement accuracy of the biological information measuring apparatus.

In addition, an interval LD1 between the first light emitting unit 350 and the second light emitting unit 380 as a plurality of light emitting units is preferably equal to or greater than 1.0 mm and equal to or less than 4.9 mm. The first light emitting unit 350 and the second light emitting unit 380 are disposed within such a range, and thus it is possible to provide the biological information measuring apparatus capable of achieving a reduction in size by a compact arrangement and an improvement in measurement accuracy by secured high light intensity. Meanwhile, the above-mentioned interval LD1 between the first light emitting unit 350 and the second light emitting unit 380 can be applied to configurations, to be described in the following modification examples and embodiments, which have a plurality of light emitting units.

### Modification Example 2 of Arrangement

Next, Modification Example 2 of the arrangement of a light emitting unit and a light receiving unit will be described with reference to FIG. 8A. In a configuration of Modification Example 2, a first light emitting unit 450 and a second light emitting unit 480 as light emitting units, and a light receiving unit 440 are mounted on a substrate 460 so as to be lined up in a row along a predetermined direction in the order of the first light emitting unit 450, the light receiving unit 440, and the second light emitting unit 480. The first light emitting unit 450, the second light emitting unit 480, and the light receiving unit 440 are disposed so that a first interval D1 which is an interval between the first light emitting unit 450 and the light receiving unit 440 and a second interval D2 which is an interval between the second light emitting unit 480 and the light receiving unit 440 are set to be different distances. In addition, a light shielding member (light shielding wall 100) as a light shielding unit is provided between the first light emitting unit 450 and the light receiving unit 440 and between the second light emitting unit 480 and the light receiving unit 440.

Specifically, the first light emitting unit 450, the light receiving unit 440, and the second light emitting unit 480 are disposed so that the second interval D2 which is a distance between an outer circumferential side 480a of the second light emitting unit 480 on the light receiving unit 440 side and an outer circumferential side 440b of the light receiving unit 440 on the second light emitting unit 480 side becomes larger than the first interval D1 which is a distance between an outer circumferential side 450b of the first light emitting unit 450 on the light receiving unit 440 side and an outer circumferential side 440a of the light receiving unit 440 on the first light emitting unit 450 side (distance between the outer circumferential sides is increased).

By this arrangement, the length of a light path between the first light emitting unit 450 and the light receiving unit 440 and the length of a light path between the second light emitting unit 480 and the light receiving unit 440 are different from each other, and a timing at which light is incident on the light receiving unit 440 from the first light emitting unit 450 and a timing at which light is incident on the light receiving unit 440 from the second light emitting unit 480 are different from each other, and thus it is possible to acquire a larger amount of biological information.

### Third Modification Example of Arrangement

Next, Modification Example 3 of the arrangement of a light emitting unit and a light receiving unit will be described with reference to FIG. 8B. In the above-described arrangement of Modification Example 2, one first light emitting unit 450 and one second light emitting unit 480 are disposed with the light receiving unit 440 interposed therebetween. In a configuration of Modification Example 3, a first light emitting unit 550 and a second light emitting unit 580 as light emitting units, and a light receiving unit 540 are mounted on a substrate 560 so as to be lined up in a row along a predetermined direction in the order of the second light emitting unit 580, the first light emitting unit 550, and the light receiving unit 540. The second light emitting unit 580 and the first light emitting unit 550 are disposed so as to be lined up, and a light shielding member 70 (light shielding wall 100) as a light shielding unit is provided between the first light emitting unit 550 and the light receiving unit 540. Therefore, in the configuration of Modification Example 3, the light emitting units and the light receiving unit are disposed so that a first interval D1 which is an interval between the first light emitting unit 550 and the light receiving unit 540 and a second interval D2 which is an interval between the second light emitting unit 580 and the light receiving unit 540 are set to be different distances.

Specifically, the second interval D2 which is a distance between an outer circumferential side 580b of the second light emitting unit 580 on the light receiving unit 540 side and an outer circumferential side 540a of the light receiving unit 540 on the second light emitting unit 580 side becomes larger than the first interval D1 which is a distance between an outer circumferential side 550b of the first light emitting unit 550 on the light receiving unit 540 side and an outer circumferential side 540a of the light receiving unit 540 on the first light emitting unit 550 side (distance between the outer circumferential sides is increased).

By this arrangement, similarly to Modification Example 2, the length of a light path between the first light emitting unit 550 and the light receiving unit 540 and the length of a light path between the second light emitting unit 580 and the light receiving unit 540 are different from each other, and a timing at which light is incident on the light receiving unit 540 from the first light emitting unit 550 and a timing at which light is incident on the light receiving unit 540 from the second light emitting unit 580 are different from each other, and thus it is possible to acquire a larger amount of biological information.

### Modification Example 4 of Arrangement

Next, Modification Example 4 of the arrangement of a light emitting unit and a light receiving unit will be described with reference to FIG. 9A. In a configuration of Modification Example 4, after a first light receiving unit 640 and a second light receiving unit 670 as light receiving units share a light emitting unit 650, one first light receiving unit 640 and one second light receiving unit 670 are respectively disposed on both sides of a light emitting unit 650 along a predetermined direction and are mounted on a substrate 660 so as to be lined up in a row. A light shielding member 70 (light shielding wall 100) as a light shielding unit is provided between the light emitting unit 650 and the first light receiving unit 640 and between the light emitting unit 650 and the second light receiving unit 670. Therefore, in the configuration of Modification Example 4, the light emitting unit and the light receiving units are disposed so that a first interval D3 which is an interval between the light emitting unit 650 and the first light receiving unit 640 and a second interval D4 which is an interval between the light emitting unit 650 and the second light receiving unit 670 are set to be substantially the same distance.

Specifically, the first interval D3 which is a distance between an outer circumferential side 650b of the light emitting unit 650 on the first light receiving unit 640 side and an outer circumferential side 640a of the first light receiving unit 640 on the light emitting unit 650 side and the first interval D4 which is a distance between an outer circumferential side 650a of the light emitting unit 650 on the second light receiving unit 670 side and an outer circumferential side 670a of the second light receiving unit 670 on the light emitting unit 650 side are substantially the same as each other.

By this arrangement, similarly to Modification Example 1, the length of a light path between the light emitting unit 650 and the first light receiving unit 640 and the length of a light path between the light emitting unit 650 and the second light receiving unit 670 are set to be substantially the same as each other, and light beams emitted from the light emitting unit 650 are incident on the first light receiving unit 640 and the second light receiving unit 670 at substantially the same time, and thus it is possible to improve an S/N ratio. That is, it is possible to improve the measurement accuracy of the biological information measuring apparatus.

### Modification Example 5 of Arrangement

Next, Modification Example 5 of the arrangement of a light emitting unit and a light receiving unit will be described with reference to FIG. 9B. In a configuration of Modification Example 5, after a first light receiving unit 740 and a second light receiving unit 770 as light receiving units share a light emitting unit 750, the light emitting unit 750, the second light receiving unit 770, and the first light receiving unit 740 are mounted on a substrate 760 in this order so as to be lined up in a row along a predetermined direction. In addition, the light emitting unit 750, the second light receiving unit 770, and the first light receiving unit 740 are disposed so that a first interval D3 which is an interval between the light emitting unit 750 and the first light receiving unit 740 and a second interval D4 which is an interval between the light emitting unit 750 and the second light receiving unit 770 are set to be different distances.

Specifically, the first interval D3 which is a distance between an outer circumferential side 750b of the light emitting unit 750 on the first light receiving unit 740 side and an outer circumferential side 740a of the first light receiving unit 740 on the light emitting unit 750 side and the second interval D4 which is a distance between an outer circumferential side 750b of the light emitting unit 750 on the second light receiving unit 770 side and an outer circumferential side 770a of the second light receiving unit 770 on the light emitting unit 750 side are different from each other. In other words, the first interval D3 is larger than the second interval D4.

By this arrangement, the length of a light path between the light emitting unit 750 and the first light receiving unit 740 and the length of a light path between the light emitting unit 750 and the second light receiving unit 770 are different from each other, and a timing at which light emitted from the light emitting unit 750 is incident on the first light receiving unit 740 and a timing at which light is incident on the second light receiving unit 770 are different from each other, and thus it is possible to acquire a larger amount of biological information.

### Second Embodiment

Next, the second embodiment of the invention will be described with reference to the accompanying drawings.

Similarly to the first embodiment described above, the biological information measuring apparatus according to the second embodiment is a heart rate monitoring apparatus which is worn on a living body (for example, a human body) of which biological information is measured and which measures biological information such as a pulse (heart rate). Meanwhile, in the following drawings, each component has a size to the extent that the component can be recognized in the drawing, and thus a description may be given by appropriately making a dimension and proportion of each component different from those of an actual component.

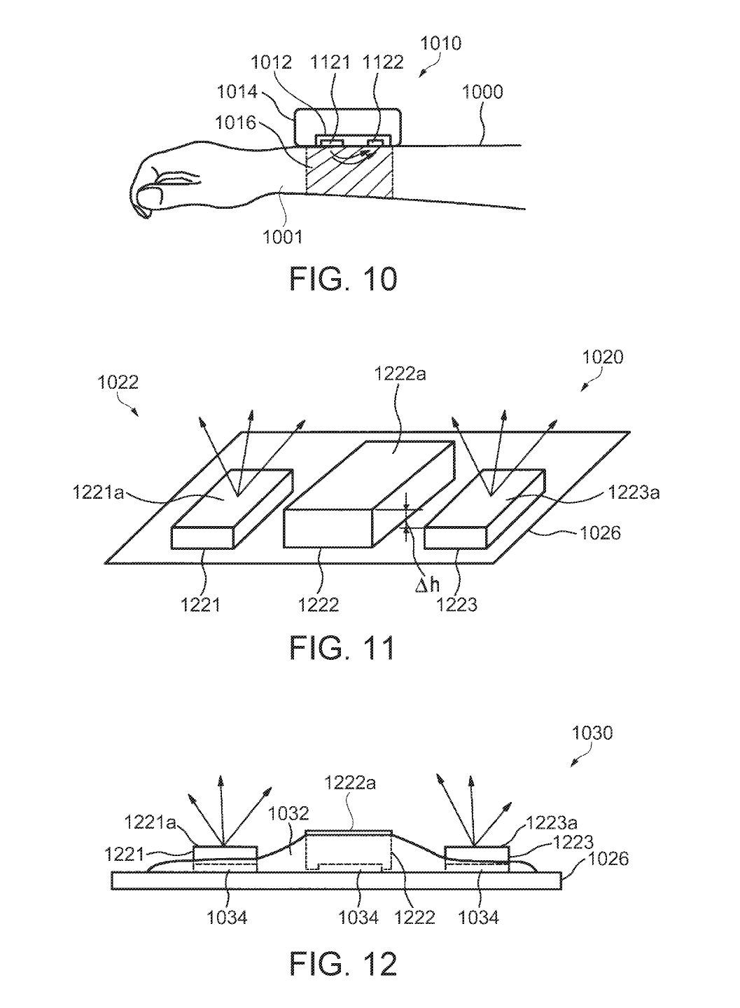

First, before a heart rate monitoring apparatus 1010 as the biological information measuring apparatus according to the second embodiment is described, an example of the art of the heart rate monitoring apparatus as the biological information measuring apparatus according to the second embodiment will be described with reference to FIG. 10.

FIG. 10 is a cross-sectional view illustrating a heart rate monitoring apparatus 1010 as a biological information measuring apparatus according to an example of the art which measures a physiologic parameter (biological information) of a user (test subject) 1000 (the user's arm is shown in the drawing) who is wearing the heart rate monitoring apparatus. The heart rate monitoring apparatus 1010 includes a sensor 1012 that measures a heart rate as at least one physiologic parameter of the user 1000, and a case 1014 that accommodates the sensor 1012. The heart rate monitoring apparatus 1010 is worn on the arm 1001 of the user 1000 by a fixation portion 1016 (for example, a band).

The sensor 1012 is a heart rate monitoring sensor that includes a light emitting element 1121 as a light emitting unit and a light receiving element 1122 as a light receiving unit which are two sensor elements and measures or monitors a heart rate. However, the sensor may be a sensor that measures one or more physiologic parameters (for example, a heart rate, blood pressure, the amount of air inhaled, skin conductivity, skin humidity, and the like). In addition, when the case 1014 includes a band-type housing, the heart rate monitoring apparatus can be used as a wristwatch type monitoring apparatus which is used in, for example, sport. Meanwhile, the case 1014 may have a shape capable of mainly holding the sensor 1012 at a desired position with respect to the user 1000, and may be able to arbitrarily accommodate more elements such as a battery, a processing unit, a display, and a user interface.

The biological information measuring apparatus of the conventional example is the heart rate monitoring apparatus 1010 for monitoring a user's heart rate. The sensor 1012 is an optical sensor constituted by the light emitting element 1121 and the light receiving element 1122. An optical heart rate monitor using the optical sensor depends on the light emitting element 1121 (LED is generally used) as a light source that exposes the skin to light. The light emitted from the light emitting element 1121 to the skin is partially absorbed by blood flowing through a blood vessel under the skin, but the rest of the light is reflected and leaves the skin. The reflected light is captured by the light receiving element 1122 (photodiode is generally used). A light reception signal from the light receiving element 1122 is a signal including information equivalent to the amount of blood flowing through the blood vessel. The amount of blood flowing through the blood vessel varies depending on pulse of the heart. In this manner, a signal on the light receiving element 1122 varies in response to the pulsation of the heart. In other words, a variation in the signal of the light receiving element 1122 is equivalent to the pulse of a heart rate. A pulse rate per unit time is counted (for example, per 10 seconds), to thereby obtain the number of beats of the heart for one minute (that is, a heart rate).

Hereinafter, a heart rate monitoring apparatus 1020 as the biological information measuring apparatus according to the second embodiment will be described with reference to FIG. 11. FIG. 11 is a perspective view illustrating a heart rate monitoring apparatus as the biological information measuring apparatus according to the second embodiment. Although not shown in FIG. 11, the heart rate monitoring apparatus 1020 as the biological information measuring apparatus according to the second embodiment is worn on a user's arm by a fixation portion such as a band, similar to the first embodiment described above.

In the heart rate monitoring apparatus 1020 as the biological information measuring apparatus according to the second embodiment, light emitting elements 1221 and 1223 as a plurality of (two in this example) light emitting units and a light receiving element 1222 as one light receiving unit are disposed so as to be lined up in a row. Specifically, a sensor 1022 (in this example, two light emitting elements 1221 and 1223 as a first light emitting unit and a second light emitting unit and the light receiving element 1222 as a light receiving unit are used as three sensor elements) which includes at least two sensor elements is provided.

The light receiving element 1222 as the light receiving unit is disposed between the two light emitting elements 1221 and 1223 as the first light emitting unit and the second light emitting unit. In addition, two light emitting elements 1221 and 1223 as the first light emitting unit and the second light emitting unit are disposed at line symmetrical positions with respect to a virtual line passing through the center of the light receiving element 1222 as the light receiving unit. The light emitting elements 1221 and 1223 and the light receiving element 1222 are disposed in such a manner, and thus it is possible to reduce dead space and to achieve space saving. In addition, light beams from both the first light emitting unit and the second light emitting unit, which are located at line symmetrical positions, gather in the light receiving unit, and thus detection can be performed more accurately.

The sensor element detects a sensor signal. The sensor 1022 includes an optical sensor constituted by the light emitting elements 1221 and 1223 using two LEDs for emitting light to the skin of a user, and at least one light receiving element 1222 (photodiode) for receiving the light reflected from the skin. Further, the heart rate monitoring apparatus 1020 includes a case or a housing (not shown). The case or the housing may be similar to or the same as the case 1014 illustrated in FIG. 10, or may be similar to or the same as the case portion 30 in the first embodiment described above.

The sensor 1022 is carried on one surface of a carrier (substrate) 1026. Here, a configuration including the carrier (substrate) 1026 and the sensor 1022 carried on the carrier (substrate) 1026 corresponds to a biological information measuring module. Meanwhile, the same is true of the third to fifth embodiments. Light emitted from the light emitting elements 1221 and 1223 can be reflected without being absorbed into the skin or the like, and can directly reach the light receiving element 1222. In the heart rate monitoring apparatus 1020, a distance between the carrier 1026 and each of upper surfaces 1221a and 1223a of the respective light emitting elements 1221 and 1223 is smaller than a distance between the carrier 1026 and an upper surface 1222a of the light receiving element 1222. That is, a difference between the distance between the carrier 1026 and each of the upper surfaces 1221a and 1223a of the respective light emitting elements 1221 and 1223 and the distance between the carrier 1026 and an upper surface 1222a of the light receiving element 1222 is Δh. The light receiving element 1222 receives light from the upper surface 1222a thereof which is the uppermost surface layer. According to these configurations, there is an effect that the most of light emitted from the light emitting elements 1221 and 1223 is directed to the skin and reflected light is directly incident on the light receiving element 1222 without going through an air layer or the like. In other words, since a structure in which the light receiving element 1222 comes into close contact with the skin is formed, a structure in which a gap is not likely to be generated between the upper surface (light receiving surface) 1222a of the light receiving element 1222 and the skin can be formed, and thus it is possible to prevent light, such as external light, which serves as a noise source from being incident on the upper surface 1222a. In addition, light from the light emitting elements 1221 and 1223 which does not pass through the skin, for example, light being directly incident on the light receiving element 1222 from the light emitting elements 1221 and 1223 cannot reach the upper surface 1222a of the light receiving element 1222.

### Third Embodiment

Next, a heart rate monitoring apparatus 1030 as the biological information measuring apparatus according to the third embodiment will be described with reference to FIG. 12. FIG. 12 is a front view illustrating a heart rate monitoring apparatus as the biological information measuring apparatus according to the third embodiment. Meanwhile, although not shown in FIG. 12, the heart rate monitoring apparatus 1030 as the biological information measuring apparatus according to the third embodiment is worn on a user's arm by a fixation portion such as a band, similar to the first embodiment described above.

As illustrated in FIG. 12, electric connection terminals 1034 of light emitting elements 1221 and 1223 as light emitting units and a light receiving element 1222 as a light receiving unit have to be preferably covered with an insulating material (for example, epoxy resin) 1032 in order to protect electrical elements. In addition, a configuration can be adopted in which the insulating material 1032 does not cover the light emitting elements 1221 and 1223 and the light receiving element 1222. Specifically, a configuration can be adopted in which the insulating material 1032 is buried in a region between the light emitting element 1221 and the light receiving element 1222 and a region between the light emitting element 1223 and the light receiving element 1222. In other words, a configuration can be adopted in which at least an upper surface 1222a of the light receiving element 1222 and upper surfaces 1221a and 1223a of the light emitting elements 1221 and 1223 are not covered with the insulating material 1032. With such a configuration, it is possible to suppress disturbance due to an air gap between the skin and the light emitting elements 1221 and 1223. Further, a configuration may be adopted in which the insulating material 1032 covers the upper surfaces 1221a and 1223a of the light emitting elements 1221 and 1223 and the upper surface 1222a of the light receiving element 1222. With such a configuration, the upper surface 1222a of the light receiving element 1222 which comes into contact with the skin and the upper surfaces 1221a and 1223a of the light emitting elements 1221 and 1223 can be protected, and thus it is possible to prevent the upper surface 1222a of the light receiving element 1222 and the upper surfaces 1221a and 1223a of the light emitting elements 1221 and 1223 from being damaged. In this case, the insulating material 1032 can be regarded as a protection film.

In the heart rate monitoring apparatus 1030 as the biological information measuring apparatus according to this third embodiment, the insulating material 1032 using an epoxy resin is provided, as an example which is generally implementable. In FIG. 12, the insulating material 1032 is disposed so as not to cover the upper surfaces 1221a and 1223a of the light emitting elements 1221 and 1223, and protects the electric connection terminals 1034. Light beams emitted from the light emitting elements 1221 and 1223 are indicated by an arrow.

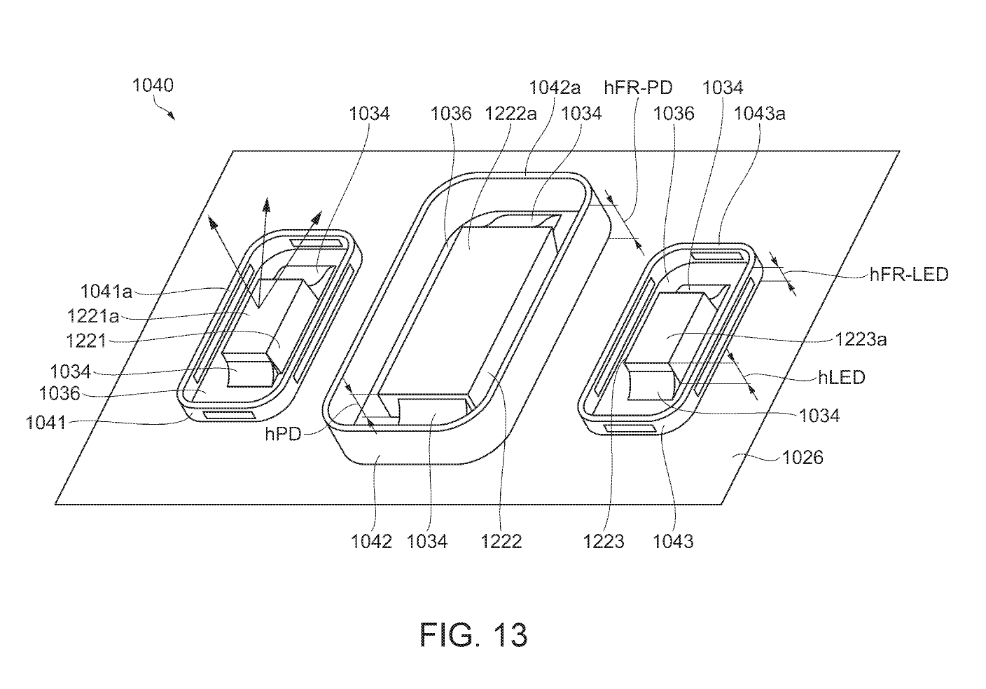

In this manner, the insulating material 1032 is minimally disposed to the extent that a correct function of the heart rate monitoring apparatus 1030 is not hindered, and thus the heart rate monitoring apparatus 1030 can be further improved by protecting the electric connection terminals 1034 of the light emitting elements 1221 and 1223 and the light receiving element 1222. Meanwhile, it is more preferable to configure a heart rate monitoring apparatus 1040 as the biological information measuring apparatus according to the fourth embodiment as illustrated in FIG. 13, instead of adopting the configuration of this third embodiment in which an epoxy resin is injected.

### Fourth Embodiment

Next, a heart rate monitoring apparatus 1040 as the biological information measuring apparatus according to the fourth embodiment will be described with reference to FIG. 13. FIG. 13 is a perspective view illustrating a heart rate monitoring apparatus as the biological information measuring apparatus according to the fourth embodiment. Meanwhile, although not shown in FIG. 13, the heart rate monitoring apparatus 1040 as the biological information measuring apparatus according to the fourth embodiment is worn on a user's arm by a fixation portion, such as a band, similar to the first embodiment described above.

In the heart rate monitoring apparatus 1040 as the biological information measuring apparatus according to the fourth embodiment, frames 1041, 1042, and 1043 created are disposed. The frames 1041, 1042, and 1043 are disposed in the vicinity of the light emitting elements 1221 and 1223 as light emitting units and the light receiving element 1222 as a light receiving unit, and a space 1036 is formed between each of the frames 1041, 1042, and 1043 and each of the light emitting elements 1221 and 1223 and the light receiving element 1222. An insulating material (not shown in FIG. 13) is injected with the frames 1041, 1042, and 1043 as guides to cover the electric connection terminals 1034 of the light emitting elements 1221 and 1223 and the light receiving element 1222.

In the example shown in the fourth embodiment, the light emitting elements 1221 and 1223 and the light receiving element 1222 are surrounded by the respective frames 1041, 1042, and 1043. Meanwhile, as another example, all of the frames 1041, 1042, and 1043 may be coupled to each other, or all of the sensor elements may be surrounded by an integrated frame. Meanwhile, the frames 1041, 1042, and 1043 can be used as light shielding walls as examples of light shielding units. The frames 1041, 1042, and 1043 are used as light shielding walls, and thus it is possible to prevent light emitted from the light emitting elements 1221 and 1223 from being directly incident on the light receiving element 1222.

As an improvement for preventing the function of the heart rate monitoring apparatus 1040 from being affected, it is preferable that upper edges 1041a and 1043a of the frames 1041 and 1043 in the vicinity of the light emitting elements 1221 and 1223 are lower than the upper surfaces 1221a and 1223a of the light emitting elements 1221 and 1223. In other words, a distance hFR-LED between the carrier 1026 and each of the upper edges 1041a and 1043a of the respective frames 1041 and 1043 is the same as or smaller than a distance hLED between the carrier 1026 and each of the upper surfaces 1221a and 1223a of the light emitting elements 1221 and 1223 which are surrounded by the respective frames 1041 and 1043 (hFR-LEDhLED).

It is preferable that a difference between the distance hLED between the carrier 1026 and each of the upper surfaces 1221a and 1223a of the respective light emitting elements 1221 and 1223 and the distance hFR-LED between the carrier 1026 and each of the upper edges 1041a and 1043a of the respective frames 1041 and 1043 is set to be in a range from 0.1 mm to 0.8 mm. Meanwhile, it is more preferable that a difference between the distance hLED between the carrier 1026 of each of the upper surfaces 1221a and 1223a of the respective light emitting elements 1221 and 1223 and the distance hFR-LED between the carrier 1026 and each of the upper edges 1041a and 1043a of the respective frames 1041 and 1043 is set to be in a range from 0.2 mm to 0.5 mm.

In addition, it is preferable that an upper edge 1042a of the frame (receiver frame) 1042 in the vicinity of the light receiving element 1222 is higher than the upper surface 1222a of the light receiving element 1222. In other words, a distance hFR-PD between the carrier 1026 and the upper edge 1042a of the frame 1042 is larger than a distance hPD between the carrier 1026 and the upper surface 1222a of the light receiving element 1222 surrounded by the frame 1042 (hFR-PD>hPD).

It is preferable that a difference between the distance hPD between the carrier 1026 and the upper surface 1222a of the light receiving element 1222 and the distance hFR-PD between the carrier 1026 and the upper edge 1042a of the frame 1042 is set to be in a range from 0 mm to 0.5 mm. Meanwhile, it is more preferable that a difference between the distance hPD between the carrier 1026 and the upper surface 1222a of the light receiving element 1222 and the distance hFR-PD between the carrier 1026 and the upper edge 1042a of the frame 1042 is set to be in a range from 0.1 mm to 0.2 mm.

Further, the distance hFR-PD between the carrier 1026 and the upper edge 1042a of the frame 1042 is larger than the distance hLED between the carrier 1026 and the upper surfaces 1221a and 1223a of the respective light emitting elements 1221 and 1223 (hFR-PD>hLED).

Meanwhile, for example, when the light receiving element 1222 and the light emitting elements 1221 and 1223 are close to each other, a configuration may be adopted in which only one frame wall is present between the light receiving element 1222 and each of the light emitting elements 1221 and 1223. This may occur because of manufacturing easiness. When the one frame wall is a case, frame walls of the frames of both the light receiving element 1222 and each of the light emitting elements 1221 and 1223 are coincident with each other. This means that the frame walls of the light emitting elements 1221 and 1223 become relatively high. In detail, the frame wall on the light receiving element 1222 side of the frames 1041 and 1043 surrounding the respective light emitting elements 1221 and 1223 become relatively high, and the other frame wall becomes lower than the upper surfaces 1221a and 1223a of the respective light emitting elements 1221 and 1223.

Further, instead of the frames 1041, 1042, and 1043, a configuration may be adopted in which a first wall portion is provided between the light receiving element 1222 and the light emitting element 1221 or the light emitting element 1223 and a second wall portion is provided on the outside of the light emitting elements 1221 and 1223, that is, on the side opposite to the first wall portion with respect to the light receiving element 1222.

In such a configuration, a distance between the carrier 1026 and the upper surface of the first wall portion may be larger than a distance between the carrier 1026 and the upper surface of the second wall portion. With such a configuration, it is possible to realize the function of the frame using a smaller number of members than in a case where a light emitting element and a light receiving element are surrounded as illustrated in FIG. 13.

Meanwhile, the frames 1041 and 1043 or the frame 1042 are used as in this fourth embodiment, and thus it is possible to prevent an insulating material to be injected, such as an epoxy resin, from flowing out. In this manner, the partitioning of an insulating material such as an epoxy resin by creating an additional structure is option of allowing high mass productivity to be obtained. Meanwhile, the frames 1041 and 1043 or the frame 1042 may be formed of the same material as that of the carrier 1026. For example, the frames may be formed by injection molding using an epoxy-based resin or a polycarbonate-based resin.

As described above, the insulating material 1032 (see FIG. 12) protects the electric connection terminals 1034 of the sensor elements (light emitting elements 1221 and 1223 and the light receiving element 1222). However, the electric connection terminals 1034 have to further come into contact with additional electronic apparatuses (for example, a driver, detection electronics, a processor, or a power supply) which are other elements. This means that there is any electrical connection between the carrier 1026 (may be a printed circuit board (PCB)) and the additional electronic apparatuses. In addition, the structure of the heart rate monitoring apparatus according to this embodiment can be applied not only to an apparatus for measuring a heart rate but also to apparatuses for measuring pulse waves and pulse.

### Fifth Embodiment

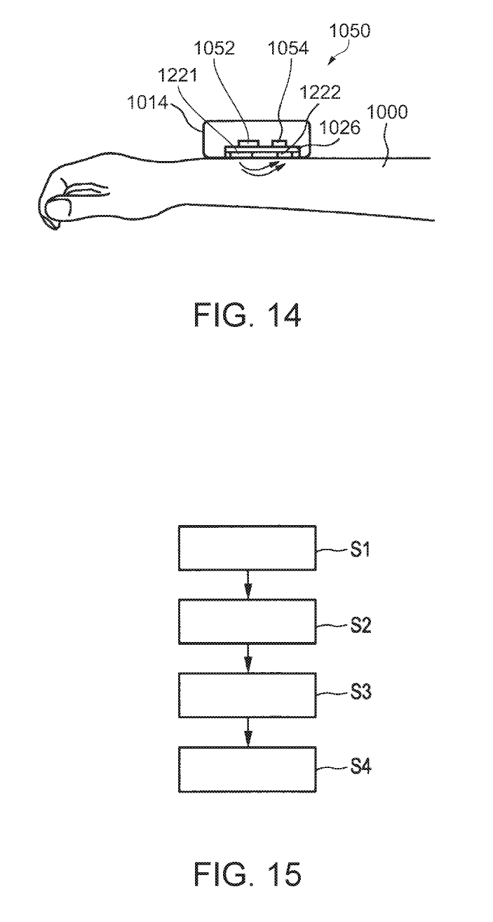

A heart rate monitoring apparatus 1050 as the biological information measuring apparatus according to the fifth embodiment will be described with reference to FIG. 14. FIG. 14 is a cross-sectional view illustrating a heart rate monitoring apparatus as the biological information measuring apparatus according to the fifth embodiment. Meanwhile, although not shown in FIG. 14, the heart rate monitoring apparatus 1050 as the biological information measuring apparatus according to the fifth embodiment is worn on a user's arm by a fixation portion such as a band, similar to the first embodiment described above.

The heart rate monitoring apparatus 1050 as the biological information measuring apparatus according to the fifth embodiment includes the above-mentioned additional electronic apparatuses (for example, a processor 1052 and a driver 1054). An external electric connection terminal (not shown) is not disposed on a carrier 1026 which is the same as that on which sensor elements (light emitting element 1221 as a light emitting unit and a light receiving element 1222 as a light receiving unit) are disposed. In other words, the additional electronic apparatuses are disposed on a carrier different from the carrier on which the sensor elements are disposed, or a substrate. With such a configuration, it is possible to mount necessary additional electronic apparatuses on the heart rate monitoring apparatus 1050 while maintaining a satisfactory contact between the skin and the sensor elements (light emitting element 1221 and the light receiving element 1222). For example, the external electric connection terminal can be disposed on the side surface of the carrier 1026.

As described above, different types of sensors can be used in the biological information measuring apparatus according to the invention. For example, when the light receiving element 1222 mentioned above is an electric sensor, two skin conductance electrodes (for example, sensor elements (the light emitting element 1221 and the light receiving element 1222 which are illustrated in FIG. 11)) which come into contact with the skin of a user and measure the conductivity of the user are covered with the skin. Meanwhile, two or more types of sensors can be used in such a type of biological information measuring apparatus, and the number of sensor elements does not matter.

In the second to fifth embodiments, a flow chart of a method of manufacturing the proposed biological information measuring apparatus that measures a physiologic parameter is illustrated in FIG. 15.

In first step S1, the sensor 1022 including at least two sensor elements (the light emitting element 1221 and the light receiving element 1222) for detecting a sensor signal is disposed on the carrier 1026. In second step S2, an electrical contact between the sensor elements is formed in the carrier 1026. In third step S3, one or more frames 1041 and 1042 are formed on the carrier 1026 in the vicinity of the sensor 1022 and/or the individual sensor elements (the light emitting element 1221 and the light receiving element 1222). In fourth step S4, the insulating material 1032 is injected into and filled in regions surrounded by the respective frames 1041 and 1042 so as not to cover the upper surfaces 1221a and 1222a of the sensor elements (the light emitting element 1221 and the light receiving element 1222) which are provided on the carrier 1026.

According to the second to fifth embodiments described above, a method of protecting an electrical contact that does not exert a bad influence on the performance of the biological information measuring apparatus is proposed. The biological information measuring apparatus is formed by such a method as that in which the performance of a sensor is maintained. For example, at least one of the frames 1041 and 1043 prevents the position of the sensor with respect to the skin from being shifted. Further, at least one of the frames 1041 and 1043 can help emitted direct light to be prevented from being input to the light receiving element 1222. It is preferable that the heights of the frames 1041 and 1043, facing the light receiving element 1222, in the vicinity of the respective light emitting elements 1221 and 1223 have to be smaller than the heights of the upper surfaces 1221a and 1223a of the respective light emitting elements 1221 and 1223. In addition, the frame 1042 in the vicinity of the light receiving element 1222 may be higher than the upper surface 1222a of the light receiving element 1222.

Also in the biological information measuring apparatuses according to the second to fifth embodiments described above, a configuration of an interval between the light emitting unit and the light receiving unit, described in the first embodiment, can be applied. With such a configuration, it is possible to obtain the same effects as those in the first embodiment.

### Sixth Embodiment

The biological information measuring apparatuses of the first to fifth embodiments described above may include various types of sensors such as, a strain gauge, a thermometer, a clinical thermometer, an acceleration sensor, a gyro sensor, a piezoelectric sensor, a pressure sensor, a sphygmomanometer, an electrochemical sensor, a global positioning system (GPS), and a vibrometer. The biological information measuring apparatuses include these sensors, and thus it is possible to derive information regarding a personal physiological state on the basis of data indicating one or one or more physiological parameters, such as heartbeat, pulse, a variation between pulsations, an elektrokardiogram (EKG), an electrocardiogram (ECG), a respiration rate, a skin temperature, a body temperature, a body heat flow, a galvanic skin response, a galvanic skin reflex (GSR), an electromyogram (EMG), an electroencephalogram (EEG), an electrooculography (EOG), blood pressure, body fat, a hydration level, an activity level, a body motion, oxygen consumption, glucose, a blood glucose level, muscle mass, pressure applied to a muscle, pressure applied to a bone, ultraviolet absorption, a sleep state, a physical condition, a stress state, and a posture (for example, lying, standing upright, and sitting). In addition, values obtained by the various types of sensors are transmitted to, for example, a portable communication terminal such as a smartphone, a mobile phone, or a feature phone, or an information processing terminal such as a computer or a tablet computer, so that the portable communication terminal or the information processing terminal may execute the arithmetic processing of the physiological parameters.

A user inputs his or her own profile to the biological information measuring apparatus, the portable communication terminal, or the information processing terminal before measuring biological information. Thereby, the user can receive user's unique characteristic information and environmental information which are required to be coped with, in order to maximize a possibility of a recommended healthy lifestyle being established and maintained, on the basis of the profile and biological information measurement results. Examples of information to be provided include one or two or more of exercise information such as an exercise type, an exercise strength, and an exercise time, meal information such as a meal time, the amount of meal, recommended intake ingredients and intake menus, and intake ingredients and intake menus that should be avoided, life support information such as a sleep time, the depth of sleep, the quality of sleep, a wake-up time, a landing time, a working time, stress information, consumed calories, intake calories, and calorie balance, physical information such as basal metabolism, the amount of body fat, a body fat percentage, and muscle mass, medication information, supplement intake information, and medical information.

Examples of the user's own profile input in advance include one or two or more of the age, the date of birth, the sex, hobbies, an occupation type, a blood type, a past sports history, an activity level, meal, the regularity of sleep, the regularity of bowel habit, situation adaptability, durability, responsiveness, the strength of reaction, user's personality such as a temper, a user's self-independence level, independent formation, self-management, sociability, a memory and an academic attainment ability, a user's awakening level, a perception speed, an ability to avoid attention alienation factors, user's attention including an awakening state and a self-supervision ability, an attention continuance ability, the weight, the height, blood pressure, a user's health state, medical examination results by a doctor, the date of a medical examination by a doctor, the presence or absence of a contact between a doctor and a health care person, medicines and supplements that are currently taken, the presence or absence of an allergy, an allergy history, the current allergy symptoms, an opinion of behavior pertaining to health, a user's disease history, a user's operation history, a family medical history, a social phenomenon, such as a divorce or unemployment, which is required to be adjusted by an individual, conviction pertaining to a user's health priority, a sense of values, an ability to change behavior, a phenomenon considered to be a cause of the stress of life, a stress management method, the degree of user's own consciousness, the degree of user's empathy, the degree of user's authority transfer, user's pride, user's exercise, a sleep state, a relaxed state, the current routine of daily activity, the personality of an important person in user's life (for example, a spouse, a friend, a colleague, or a superior officer), and a user's way to catch whether a conflict that disturbs a healthy lifestyle or contributes to stress is present in a relationship with an important person.

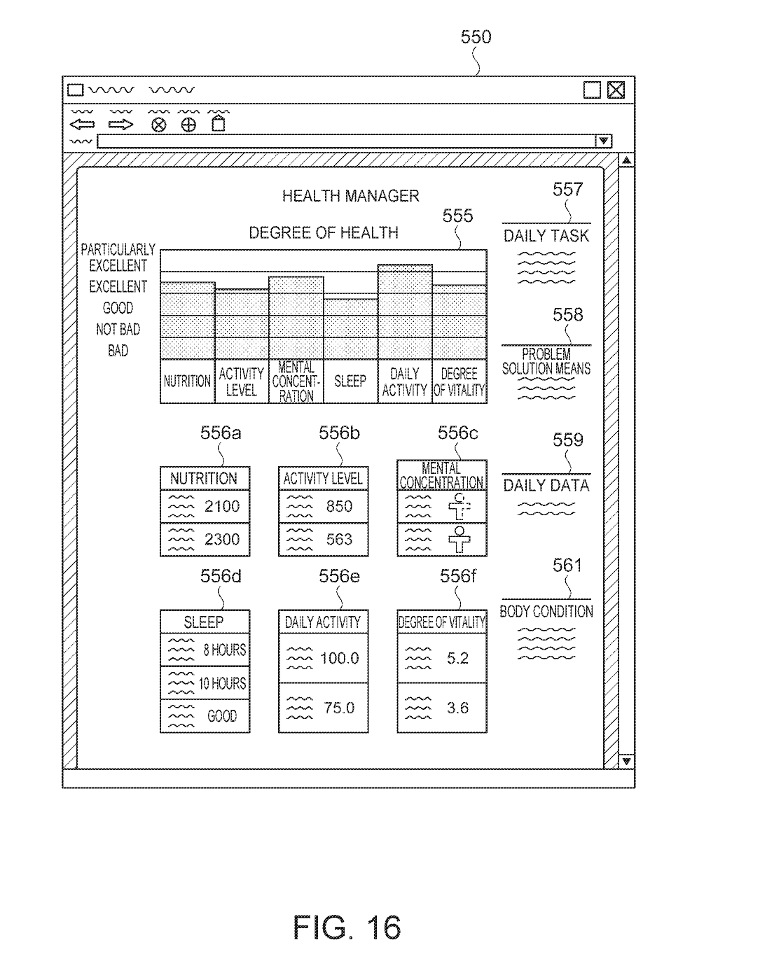

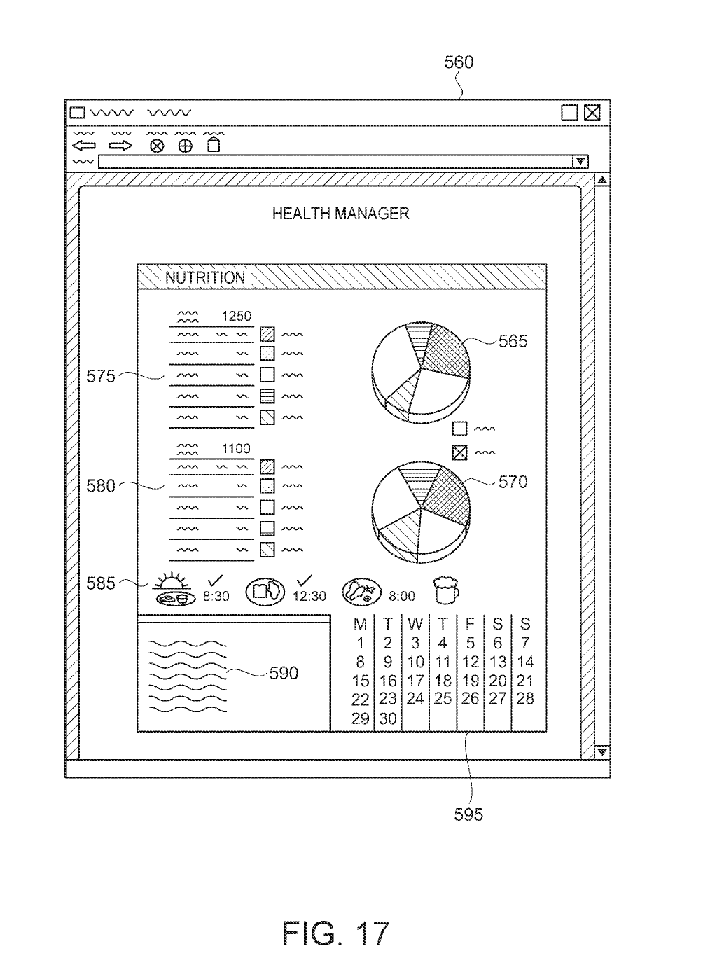

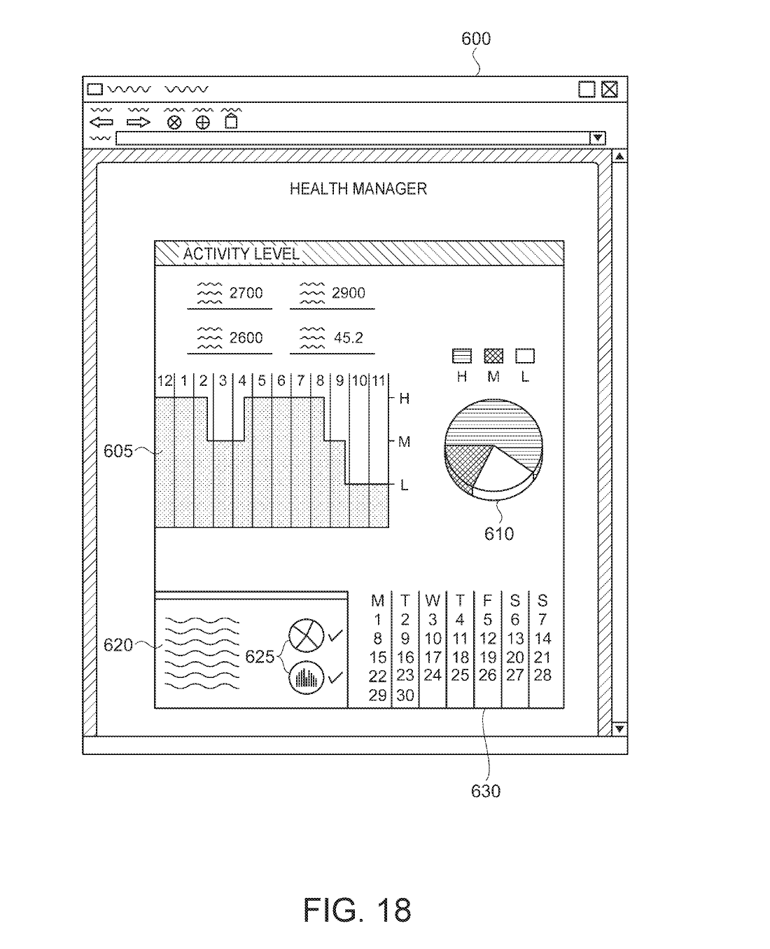

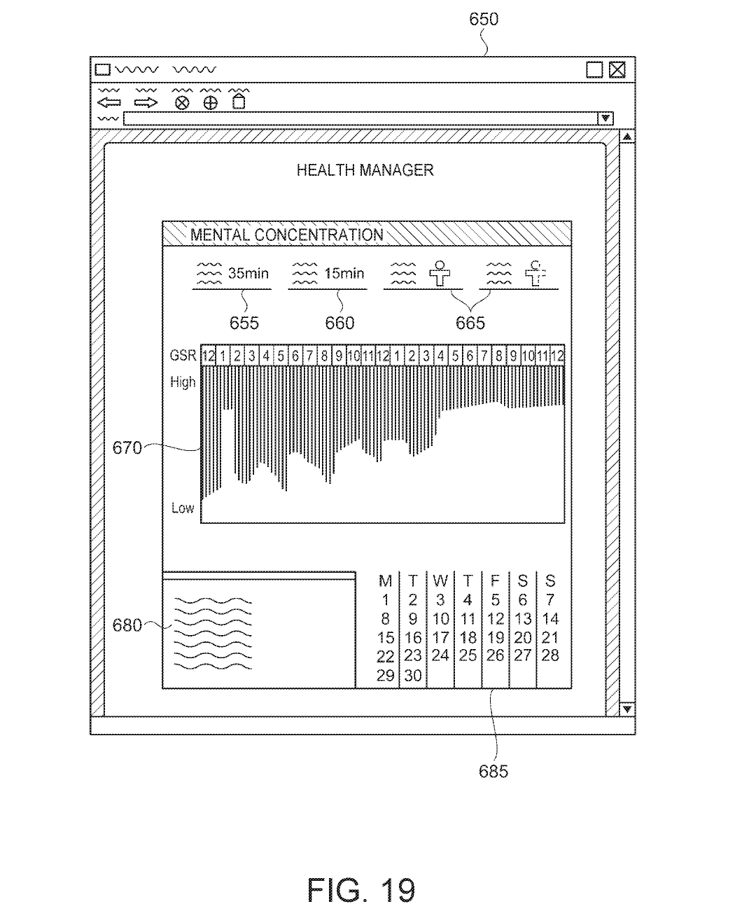

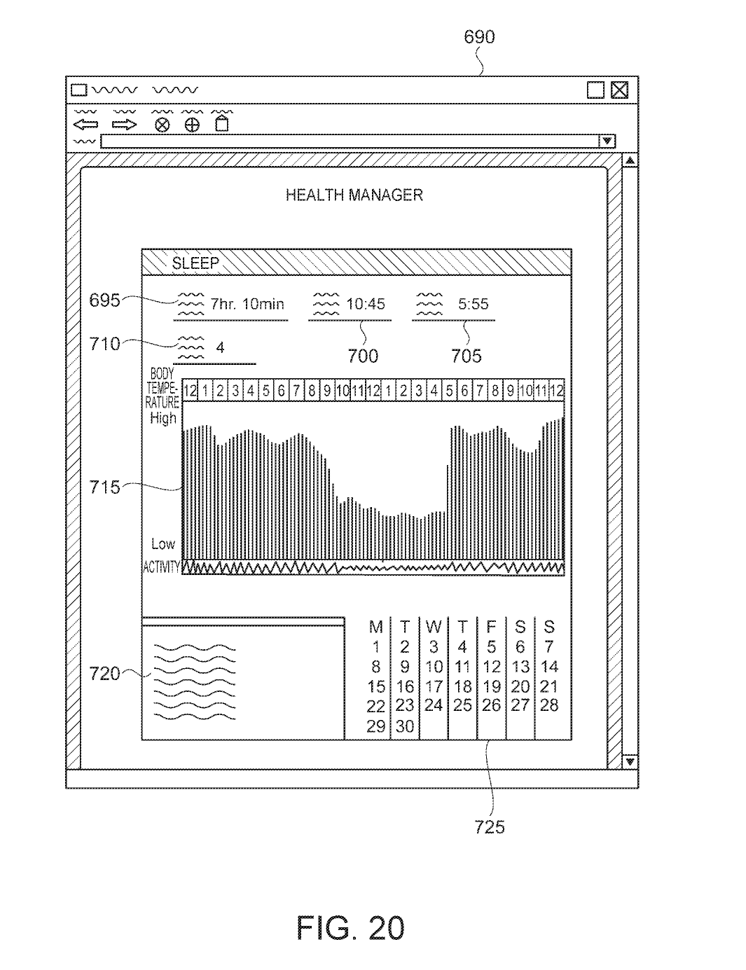

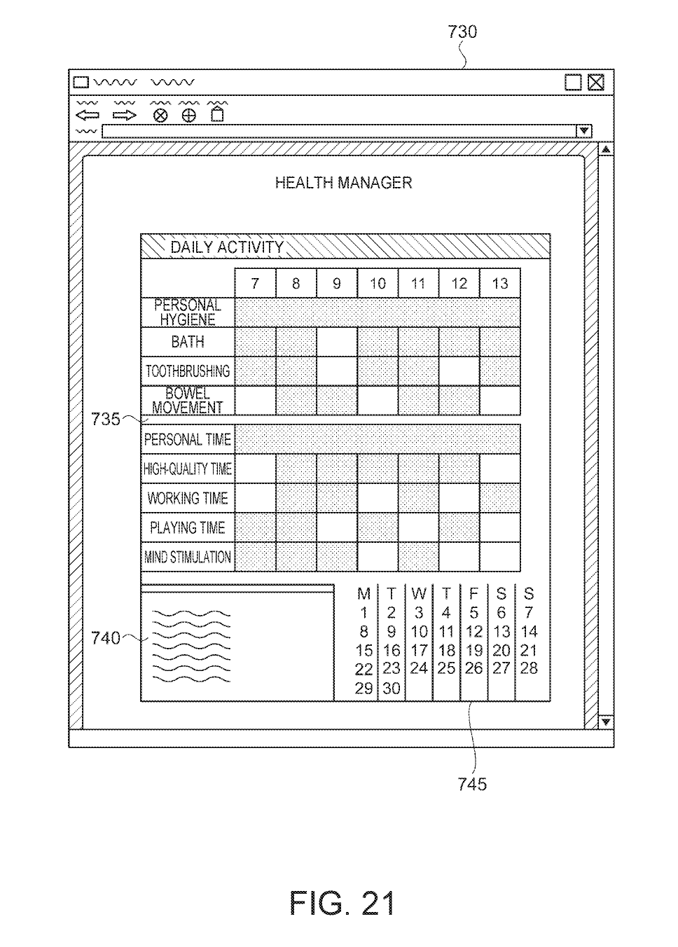

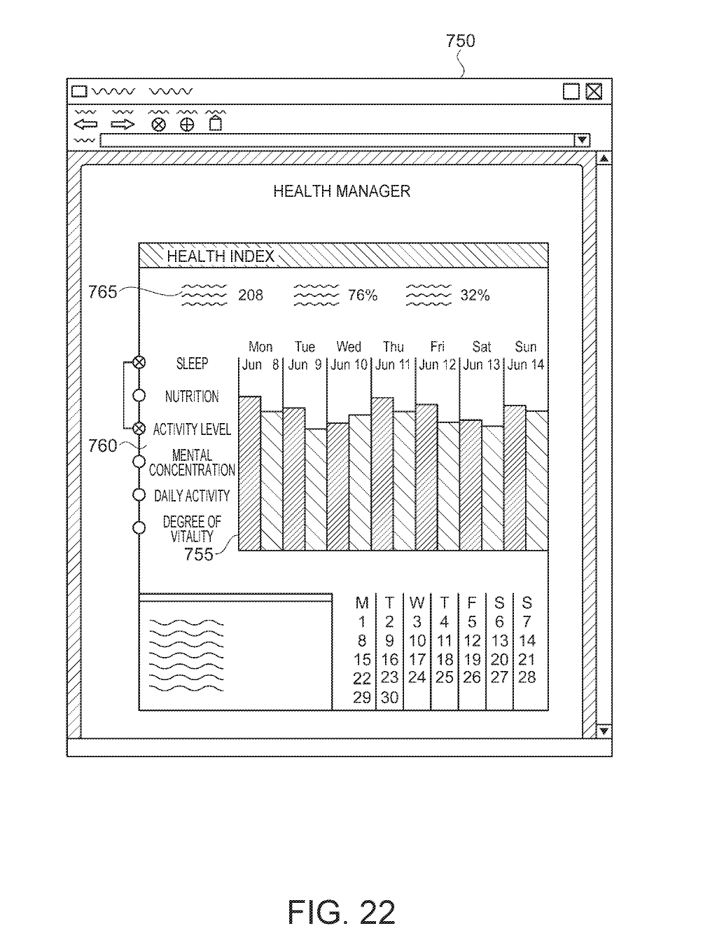

Here, reference will be made to FIGS. 16 to 22 to describe a biological information measuring apparatus according to a sixth embodiment which is capable of receiving user's unique characteristic information and environmental information which are required to be coped with, in order to maximize a possibility of a recommended healthy lifestyle being established and maintained. FIG. 16 is a schematic diagram illustrating a web page serving as a starting point of a health manager in the biological information measuring apparatus of the sixth embodiment. FIG. 17 is a diagram illustrating an example of a nutrition web page, and FIG. 18 is a diagram illustrating an example of an activity level web page. In addition, FIG. 19 is a diagram illustrating an example of a mental concentration web page, and FIG. 20 is a diagram illustrating an example of a sleep web page. In addition, FIG. 21 is a diagram illustrating an example of a daily activity web page, and FIG. 22 is a diagram illustrating an example of a health degree web page.

Although not shown in the drawing, the biological information measuring apparatus according to the sixth embodiment includes, for example, a sensor device which is connected to a microprocessor. In the biological information measuring apparatus according to the sixth embodiment, pieces of data regarding various life activity items which are finally transmitted to a monitor unit and stored, and personal data or living information which is input by a user from a website maintained by the monitor unit are processed by the microprocessor and are provided as biological information. Hereinafter, a specific example will be described.

A user has access to a health manager for the user through a web page, application software, and other communication media. FIG. 16 illustrates a web page 550 serving as a starting point of the health manager, as an example. In the web page 550 of the health manager shown in FIG. 16, various pieces of data are provided to a user. The provided data is one or more pieces of data of, for example, (1) data indicating various physiological parameters based on values measured by various sensor devices, (2) data derived from data indicating various physiological parameters, and (3) data indicating various context parameters generated by the sensor device and data input by the user.

Analysis state data has features that a certain utility or algorithm is used in order to perform conversion into (1) data indicating various physiological parameters acquired by the sensor device, (2) data derived from various physiological parameters, (3) the degree of health obtained by calculating one or more pieces of data of data indicating various context parameters acquired by the sensor device and data input by the user, (4) the degree of good health and a lifestyle index, and the like. For example, it is possible to calculate the amounts of calories, protein, fat, carbohydrates, and certain vitamin on the basis of data input by the user in relation to food taken. In addition, as another example, it is possible to provide indexes of stress levels over a desired period of time to the user by using a skin temperature, a heart rate, a respiration rate, a heat flow and/or a GSR. As still another example, it is possible to provide indexes of sleep patterns over a desired period of time to the user by using a skin temperature, a heat flow, a variation between pulsations, a heart rate, pulse, a respiration rate, a central body temperature, a galvanic skin response, an EMG, an EEG, an EOG, blood pressure, oxygen consumption, ambient sounds, and body motion detected by a device such as an accelerometer.

In the web page 550 illustrated in FIG. 16, a health index 555 as the degree of health is displayed. The health index 555 is a graphic utility for measuring the degree of achievement of user's results and a recommended healthy daily task and giving feedback to member users. In this manner, the health index 555 indicates health states and progress conditions of action pertaining to health maintenance of the member users. The health index 555 includes six categories regarding the health and lifestyle of a user, that is, nutrition, an activity level, mental concentration, sleep, daily activity, and the degree of vitality (overall impression). The category of “nutrition” pertains to information regarding what, when, and how much the person (user) has eaten and taken. The category of “activity level” pertains to the amount of exercise regarding how much the person has moved around. The category of “mental concentration” pertains to the quality (ability) of the activity for making the person (user) set to be in a relaxed state in a state where the mind of the person is in a highly concentrated state, and to a period of time for which the person concentrates on the activity. The category of “sleep” pertains to the quality and amount of sleep of the person (user). The category of “daily activity” pertains to matters that have to be performed every day by the person (user) and to health risks that the person meets with. The category of “the degree of vitality (impression)” pertains to a general way to catch whether being in a good mood on a certain day. Preferably, each of the categories includes a level display or a bar graph indicating how many results the user has attained on a scale varying between “bad” and “good”.

When each member user terminates the above-mentioned initial examination, a profile for providing a user's own characteristics and a summary of a living environment to the user is created, and recommended healthy daily tasks and/or targets are presented. The recommended healthy daily tasks include any combination in specific pieces of advice regarding appropriate nutrition, exercise, mental concentration, and user's daily activity (life). A model schedule or the like may be presented as a guide indicating how to take activity items pertaining to the recommended healthy daily tasks in the user's life. The user is regularly subjected to the examination, and practices the above-mentioned items accordingly on the basis of the results thereof.

The category of “nutrition” is calculated from both data input by a user and data sensed by a sensor device. The data input by the user includes the times for breakfast, lunch, and dinner, and any snack and the eating and drinking times thereof, and food to be eaten and drunk, supplements such as vitamin, and water or another liquid (drinking water or liquid food) which is drunk during a time which is selected in advance. A central monitoring unit calculates consumed calories or well-known nutritional values such as the contents of protein, fat, carbohydrates, vitamin, and the like, on the basis of the data and stored data regarding known characteristics of various articles of food.

In the category of “nutrition”, a recommended healthy daily task can be determined on the basis of the bar graph indicating the nutrition of the health index 555. The recommended healthy daily task can be adjusted on the basis of information such as the sex, age, and height/weight of a user. Meanwhile, a user or a representative of the user can set a target of certain nutrition pertaining to the amount of calories consumed every day, the amount of nutriments such as protein, fiber, fat, and carbohydrates, the amount of water, and ratios thereof to the total intake. Parameters used for the calculation of the bar graph include the number of meals for one day, the amount of water consumed, and the type and amount of food eaten every day which are input by a user.

Nutritional information is presented to a user by a nutrition web page 560 as illustrated in FIG. 17. It is preferable that the nutrition web page 560 includes nutrition numerical charts 565 and 570 that are pie charts showing actual and target numerical values of nutrition, and nutrition intake charts 575 and 580 showing an actual total nutrition intake amount and a target total nutrition intake amount. In the nutrition numerical charts 565 and 570, it is preferable that items such as carbohydrates, protein, and fat are expressed by percentage. In the nutrition intake charts 575 and 580, it is preferable that a total value and a target value of calories are expressed by being divided into ingredients such as fat, carbohydrates, protein, and vitamin. The web page 560 includes a history 585 indicating the times when food and water are consumed, a hyperlink 590 that allows a user to be able to directly check a news story pertaining to nutrition, advice for improving a daily task pertaining to nutrition, and any related advertisement on a network, and a calendar 595 in which an application period and the like can be selected. Items indicated by the hyperlink 590 can be selected on the basis of information learned from an individual through examination, and the individual's results measured by the health index.

The category of “activity level” in the health index 555 is designed so as to support a user's check regarding when and how the user had activity (moved) on that day, and the like, and both data input by the user and data sensed by the sensor device are used. The data input by the user includes details pertaining to the user's daily activity such as, for example, doing work at the desk from 8 a.m. to 5 p.m. and taking an aerobic lesson from 6 p.m. to 7 p.m. The related data sensed by the sensor device includes a heart rate, an exercise sensed by a device such as an accelerometer, a heat flow, a respiration rate, the amount of calories consumed, a GSR, and a water supply level, and these can be taken out by the sensor device or the central monitoring unit. The amount of calories consumed can be calculated by various methods such as multiplication of the type of exercise which is input by the user and the duration of exercise which is input by the user, multiplication of the sensed exercise, an exercise time, and a filter constant, or multiplication of the sensed heat flow, the time, and a filter constant.

In the category of “activity level”, a recommended healthy daily task can be determined on the basis of the bar graph indicating the activity level of the health index 555. The recommended healthy daily task includes a minimum target calories consumed by the activity, and the like. Meanwhile, the minimum target calories can be set on the basis of information such as the sex, age, height, and weight of a user. Parameters used for the calculation of the bar graph includes a time input by the user and/or a time sensed by the sensor device which are times spent for various types of exercises or an energetic lifestyle activity, and the amount of calories burned over an energy consumption parameter which is calculated in advance.

Information regarding the activity (movement) of an individual user is presented to the user by an activity level web page 600 illustrated in FIG. 18. The activity level web page 600 includes an activity degree graph 605, having a bar graph shape, which shows the user's activity monitored according to three categories, that is, “high”, “medium”, and “low” that are classified with respect to a predetermined unit time. An activity percentage chart 610 having a pie chart shape can be presented in order to express a percentage for a predetermined period of time such as, for example, one day which is spent in each of the categories by the user. In addition, the activity level web page 600 may include a calorie display (not shown) for displaying items such as a total amount of calories burned, a target value of daily burned calories, a total value of calories taken, and an aerobic exercise time. The activity level web page 600 includes at least one hyperlink 620 in order to allow the user to be able to directly check a related news story, advice for improving a daily task pertaining to an activity level, and a related advertisement on a network.

The activity level web page 600 can be viewed in various formats, and can be configured such that a user can select a bar graph, a pie chart, or both the graph and the chart and the selection can be performed by an activity level check box 625. An activity level calendar 630 is provided so that an application period and the like can be selected. Items indicated by the hyperlink 620 can be selected on the basis of information extracted from an individual through examination, and the results measured by the health index.

The category of “mental concentration” in the health index 555 is designed so as to support a user's monitoring of parameters pertaining to a time when the activity for allowing the user's body to reach a deep relaxed state while concentrating his or her mind is performed, and is based on both data input by the user and data sensed by the sensor device. In detail, the user can input a starting time and a termination time of a relaxation activity such as yoga or meditation. The quality of these activity items determined by the depth of mental concentration can be measured by monitoring parameters including a skin temperature, a heart rate, a respiration rate, and a heat flow which are sensed by the sensor device. It is also possible to use a variation in the percentage of a GSR obtained by either of the sensor device or the central monitoring unit.

In the category of “mental concentration”, a recommended healthy daily task can be determined on the basis of the bar graph indicating the activity level of the mental concentration in the health index 555. The recommended healthy daily task is displayed inclusive of daily joining in the activity of deeply relaxing a body while making mind set to be in a highly concentrated state. Parameters used for the calculation of the bar graph include the length of time spent for the mental concentration activity, the depth of the mental concentration activity, or a variation in the percentage of a skin temperature, a heart rate, a respiration rate, a heat flow, or a GSR which is sensed by the sensor device from a base line indicating quality.

Information regarding time spent for an action of deeply looking back oneself (introspection) and for mental concentration activity such as deep relaxation of a body is presented to a user by a mental concentration web page 650 illustrated in FIG. 19. Meanwhile, the mental concentration activity may be referred to as a session. The mental concentration web page 650 includes a time 655 spent for the session, a target time 660, comparison portions 665 indicating a target value of the depth of mental concentration and an actual value, and a histogram 670 indicating the overall stress level which is derived from a skin temperature, a heart rate, a respiration rate, a heat flow, and/or a GSR.

In the comparison portion 665, the contour of a human indicating a target mental concentration state is shown by a solid line, and the contour of a human indicating an actual mental concentration state varies between a blurred state (shown by a dashed line in FIG. 19) and a solid line in accordance with the level of mental concentration. In addition, the preferable mental concentration web page 650 includes a hyperlink 680 that allows a user to be able to directly check a related news story, advice for improving a daily task pertaining to mental concentration, and a related advertisement on a network, and a calendar 685 in which advice for improving a daily task pertaining to mental concentration, a related advertisement and an application period can be selected. Items indicated by the hyperlink 680 can be selected on the basis of results measured by information learned from an individual through examination, and the results measured by the health index.

The category of “sleep” in the health index 555 is designed so as to be able to support a user's monitoring of a sleep pattern and the quality of sleep. This category is intended to help a user to learn the importance of sleep in a healthy lifestyle and the relation of sleep to a daily cycle which is an ordinary daily variation in the function of the body. The category of “sleep” is based on both data input by the user and data sensed by the sensor device. The data input by the user between related time intervals includes ranks of a sleep-onset time and a wake-up time (sleep time) of the user and the quality of sleep. The related data obtained by the sensor device includes a skin temperature (body temperature), a heat flow, a variation between pulsations, a heart rate, a pulse rate, a respiration rate, a central body temperature, a galvanic skin response, an EMG, an EEG, an EOG, blood pressure, and oxygen consumption. In addition, ambient sounds and body motion which is detected by a device such as an accelerometer also have relevance. Thereafter, a sleep-onset time, a wake-up time, the interruption of sleep, the quality of sleep, the depth of sleep, and the like can be calculated and derived using the data.

The bar graph showing the sleep in the health index 555 displays a healthy daily task including the securing of a preferable nightly minimum sleep time, a predictable bedtime, and a wake-up time. Specific parameters enabling the calculation of the bar graph include a daily sleep time and a wake-up time which are sensed by the sensor device or input by the user, and the quality of sleep which is graded by the user or derived from another data.

Information regarding the sleep is presented to a user by a sleep web page 690 illustrated in FIG. 20. The sleep web page 690 includes a sleep time display 695 based on either of data from the sensor device or data input by the user, a user bedtime display 700, and a wake-up time display 705. Meanwhile, the quality of sleep which is input by the user can be displayed using a sleep quality rank 710. In addition, when a display exceeding a time interval for one day is performed in the sleep web page 690, the sleep time display 695 can be displayed as a cumulative value, and the bedtime display 700, the wake-up time display 705, and the sleep quality rank 710 can be calculated and displayed as average values. In addition, the sleep web page 690 also includes a sleep graph 715 selectable by a user who calculates and displays one sleep-related parameter during a predetermined time interval. FIG. 20 illustrates a variation in a heat flow (body temperature) for one day. The heat flow tends to be reduced while asleep and to be increased while awake. It is possible to obtain a biorhythm of the person from the information.

In addition, the sleep graph 715 displays data from an accelerometer embedded in the sensor device that monitors body motion. In addition, the sleep web page 690 can include a hyperlink 720 that allows a user to be able to directly check a news story pertaining to sleep, advice for improving a daily task pertaining to sleep, and a related advertisement on a network, and a sleep calendar 725 for selecting a related time interval. Items indicated by the hyperlink 720 can be particularly selected on the basis of information learned from an individual in examination, and results measured by the health index.

The category of “daily activity” in the health index 555 is designed so as to be able to support a user's monitoring of a certain activity, pertaining to health or safety, and risk, and is completely based on data input by a user. The category of “daily activity” pertaining to activity in a daily life includes four categories which are subordinate concepts. Specifically, the category is classified into (1) an item pertaining to personal hygiene which enables a user's monitoring of dental care using a toothbrush or floss or activity such as taking a shower, (2) an item pertaining to health maintenance which enables tracing of whether a user is taking medicine or a supplement as prescribed, and enables a user's monitoring of the consumption of cigarettes or alcohol, and the like, (3) an item pertaining to personal time which enables a user's monitoring of time or leisure, which is spent with the user's family or friend, and mental concentration activity, and (4) an item pertaining to responsibility which enables a user's monitoring of work, such as household chores, and household activity.

In the category of “daily activity”, it is preferable that the bar graph indicating the “daily activity” in the health index 555 displays the following recommended healthy daily tasks. As an example of a daily task pertaining to the personal hygiene, it is preferable that a user takes a shower or takes a bath every day, keeps his or her teeth clean by using a toothbrush or floss every day, and has regular bowel movements. In addition, as an example of a daily task pertaining to the health maintenance, it is preferable that a user takes medicine, vitamin pills, and/or supplements, does not smoke, drinks in moderation, and monitors his or her health every day by a health manager. As an example of a daily task pertaining to the personal time, it is preferable that a user makes at least predetermined time every day in order to spend the time with his or her family, and/or spends high-quality time with his or her friend, reduces time for work, takes time for leisure or play, and performs activity using his or her brain. As an example of a daily task pertaining to the responsibility, it is preferable that a user does household chores, is not late for work, and keeps a promise. The bar graph is determined by information input by a user, and/or is calculated on the basis of the degree to which the user completes activity listed up every day.

Pieces of information regarding these activity items are presented to a user by a daily activity web page 730 illustrated in FIG. 21. An activity chart 735 in the daily activity web page 730 shows whether a user has executed necessary activity by the daily task. In the activity chart 735, one or more of the subordinate concepts can be selected. In the activity chart 735, a box which is colored or shaded indicates that a user has executed necessary activity, and a box which is not colored or shaded indicates that the user has not executed the activity. The activity chart 735 can be created at a selectable time interval and can be viewed. FIG. 21 illustrates the categories of personal hygiene and personal time in a specific week as an example. Further, the daily activity web page 730 may include a hyperlink 740 that allows a user to be able to directly check a related news story, advice for improving a daily task pertaining to activity in a daily life, and a related advertisement on a network, and a daily activity calendar 745 for selecting a related time interval. Items indicated by the hyperlink 740 can be selected on the basis of information learned from an individual in examination, and results determined by the health index.

The category “the degree of vitality” in the health index 555 is designed so as to enable a user's monitoring of recognition of whether being in good spirits on a specific day, and is based on essentially subjective grade information which is directly input by the user. The user performs ranking using scales of, preferably, 1 to 5 with respect to the following nine areas, that is, (1) mental keenness, (2) the degree of mental and psychological happiness, (3) an energy level, (4) a capacity for stresses of life, (5) the degree of being concerned about appearances, (6) the degree of physical happiness, (7) self-control, (8) a motive, and (9) comfort by a relationship with others. These degrees (grades) are averaged to be used for the calculation of the bar graph of the health index 555.

FIG. 22 illustrates a vitality degree web page 750. The vitality degree web page 750 allows a user to be able to check the degree of vitality during a time interval, selectable by the user, which includes continuous or discontinuous arbitrary days. Meanwhile, in the example illustrated in FIG. 22, the degree of vitality is displayed as a health index. In the vitality degree web page 750, a user can perform selection for checking a vitality degree bar graph 755 with respect to one category or can compare the vitality degree bar graphs 755 in parallel with respect to two or more categories by using the vitality degree selection box 760. For example, the user may set only a bar graph for sleep to be in an operation state in order to check whether the overall grade of sleep has been improved compared to the previous month, or may compare the grade of sleep with the grade of an activity level corresponding thereto and evaluates the grades by simultaneously displaying the sleep and the activity level and may check whether there is some correlation between the days. The grade of nutrition and the grade of the degree of vitality may be displayed for a predetermined time interval so that it is checked whether there is some correlation between a daily dietary habit, a dietary habit during the interval, and the degree of vitality. FIG. 22 illustrates comparison between sleep and an activity level during a week from June 8 to June 14 using bar graphs, as an example for description. In addition, the vitality degree web page 750 also includes a tracing calculator 765 that displays access information, such as the sum of days in which a user has logged on and used the health manager, the proportion of days in which the user has used the health manager since admission, and the proportion of hours for which the user has used the sensor device in order to collect data, and statistics.

An example of the web page 550 serving as a starting point of the health manager illustrated in FIG. 16 includes summaries 556a to 556f of a plurality of categories, selectable by a user, which correspond to the categories of the health index 555 as the degree of health. Each of the summaries 556a to 556f of the respective categories presents a sub set of data which is selected in advance with respect to the corresponding category and is filtered. The summary 556a of the category of nutrition indicates a daily target value and an actual value of a caloric intake. The summary 556b of the category of activity level indicates a daily target value and an actual value of the amount of calories burned. The summary 556c of the category of mental concentration indicates a target value and an actual value of the depth of mental concentration. The summary 556d of the category of sleep indicates a target sleep time, an actual sleep time, and the grade of the quality of sleep. The summary 556e of the category of daily activity displays a target point and an actual point based on a ratio of completed activity to a recommended healthy daily task (daily activity). The summary 556f of the category of the degree of vitality indicates a target grade and an actual grade of the degree of health of the day.

In addition, the web page 550 may also include a hyperlink (not shown) to a news story, comments (not shown) to a user based on a tendency such as malnutrition which is checked by the first examination, and a signal (not shown). The web page may also include a daily task portion 557 that provides information to a user every day. As comments of the daily task portion 557, for example, a water intake required every day, advice for specific means for enabling the intake of water, and the like can be displayed. In addition, the web page 550 may include a problem solution section 558 that actively evaluates a user's results in each category of the health index 555 and presents advice for improvement. For example, when a user's sleep level is “low” by a system and it is suggested that the user has insomnia, the problem solution section 558 can advise a method for improving sleep. In addition, the problem solution section 558 may include the user's question regarding an improvement in results. In addition, the web page 550 may include a daily data section 559 that starts up an input dialogue box. The user can easily input various pieces of data required by the health manager, using the input dialogue box. As known in the art, the input of data can be selectively performed between the input in a list presented in advance and the input in a general free text format. In addition, the web page 550 may include a body condition section 561 that gives information regarding life symptoms such as the height and weight of a user, a body measurement value, a BMI, a heart rate, blood pressure, or any physiological parameter.

### Modification Example of Light Receiving Unit

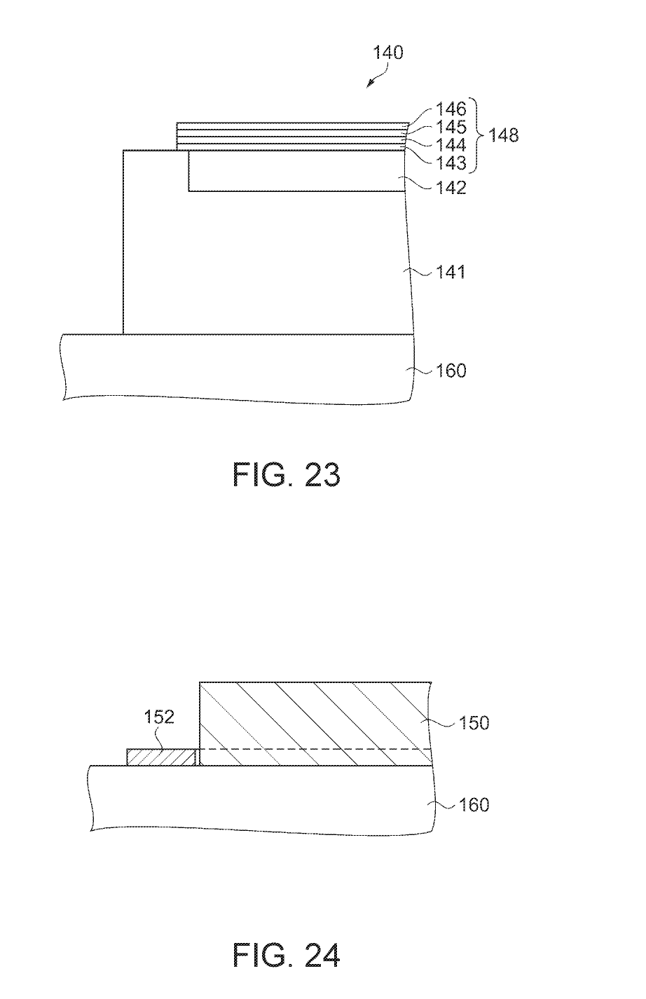

Here, a modification example of the light receiving unit 140 mentioned above will be described with reference to FIG. 23. FIG. 23 is a partial cross-sectional view illustrating a modification example of a light receiving unit. As illustrated in FIG. 23, a light receiving unit 140 mounted on a substrate 160 (sensor substrate) can be realized by a diode element 142 of a PN junction which is formed on a semiconductor substrate 141, and the like. In this case, an angle limiting filter for narrowing a light reception angle or a wavelength limiting filter (optical filter film) 148 that limits a wavelength of light incident on a light receiving element may be formed on the diode element 142. Meanwhile, for example, the wavelength limiting filter (optical filter film) 148 can be configured such that a first oxide film 143, a first nitride film 144, a second oxide film 145, and a second nitride film 146 are formed from the diode element 142 side in this order. Meanwhile, this modification example of the light receiving unit can be applied to any of the above-described embodiments.

With such a configuration, it is possible to provide the wavelength limiting filter (optical filter film) 148 in a smaller region and to provide a smaller-sized biological information measuring module and biological information measuring apparatus.

### Modification Example of Light Emitting Unit

Next, a modification example of the light emitting unit 150 mentioned above will be described with reference to FIG. 24. FIG. 24 is a partial cross-sectional view illustrating a modification example of a light emitting unit. As illustrated in FIG. 24, a reflective functional layer 152 that reflects light emitted in a peripheral direction from a light emitting unit 150 is provided in the vicinity of the light emitting unit 150 mounted on a substrate 160 (sensor substrate). Meanwhile, the reflective functional layer 152 may be provided so as to surround the vicinity of the light emitting unit 150 over the whole periphery or may be provided in at least a portion of the vicinity of the light emitting unit 150 in a plan view when seen from the upper surface side of the substrate 160. Meanwhile, this modification example of the light receiving unit can be applied to any of the above-described embodiments.

With such a configuration, light emitted in a peripheral direction of the light emitting unit 150 can be made to be reflected by a reflective functional layer 152 and to be directed to a measurement object. Thereby, it is possible to increase the intensity (light emission intensity) of light directed to the measurement object, and to improve and stabilize the measurement accuracy of biological information.

Meanwhile, embodiments of the invention have been described above in detail, but those skilled in the art may easily understand that many variations are conceivable to the extent that they do not substantially depart from the novel items and effects of the invention. Therefore, such variations all fall within the scope of the invention. For example, a term described at least once in the specification or the drawings with a different term having a broader meaning or the same meaning can be replaced with the different term anywhere in the specification or the drawings. Further, the configuration and action of each of the biological information measuring module, the light detection unit, the biological information measuring apparatus, and the like are not limited to those described in this embodiment of the invention, and a variety of changes can be made thereto.

What is claimed is:

1. A biological information measuring module comprising:
a light emitting unit that emits light to an object; and
a light receiving unit that receives light which is reflected by the object,
wherein an interval between the light emitting unit and the light receiving unit is equal to or greater than 0.4 mm and equal to or less than 1.7 mm.

2. The biological information measuring module according to claim 1, wherein an interval between the light emitting unit and the light receiving unit is equal to or greater than 0.6 mm and equal to or less than 1.3 mm.

3. The biological information measuring module according to claim 1, wherein an interval between the light emitting unit and the light receiving unit is equal to or greater than 0.8 mm and equal to or less than 1.0 mm.

4. The biological information measuring module according to claim 1, further comprising:
a substrate,
wherein the light emitting unit and the light receiving unit are supported by the substrate.

5. The biological information measuring module according to claim 1, wherein a reflective functional layer that reflects light emitted from the light emitting unit is provided in at least a portion of a vicinity of the light emitting unit.

6. The biological information measuring module according to claim 1, wherein a light shielding unit is provided between the light emitting unit and the light receiving unit.

7. The biological information measuring module according to claim 6, wherein the light shielding unit is provided so as to include at least one of a resin or a metal.

8. The biological information measuring module according to claim 1, wherein an optical filter film is provided in a light receiving region of the light receiving unit.

9. The biological information measuring module according to claim 1, wherein a light condensing unit that condenses light emitted from the light emitting unit is provided between the light emitting unit and the object.

10. The biological information measuring module according to claim 1, wherein a plurality of the light emitting units are provided.

11. The biological information measuring module according to claim 10,
wherein the plurality of light emitting units include a first light emitting unit and a second light emitting unit, and
wherein an interval between the first light emitting unit and the light receiving unit and an interval between the second light emitting unit and the light receiving unit are the same as each other.

12. The biological information measuring module according to claim 10,
wherein the plurality of light emitting units include a first light emitting unit and a second light emitting unit, and
wherein an interval between the first light emitting unit and the light receiving unit and an interval between the second light emitting unit and the light receiving unit are different from each other.

13. The biological information measuring module according to claim 10, wherein an interval between the first light emitting unit and the second light emitting unit is equal to or greater than 1.0 mm and equal to or less than 4.9 mm.

14. A biological information measuring apparatus comprising the biological information measuring module according to claim 1.

15. A biological information measuring apparatus comprising the biological information measuring module according to claim 2.

16. A biological information measuring apparatus comprising the biological information measuring module according to claim 3.

17. A biological information measuring apparatus comprising the biological information measuring module according to claim 4.

18. A biological information measuring apparatus comprising the biological information measuring module according to claim 5.

19. A biological information measuring apparatus comprising the biological information measuring module according to claim 6.

20. A biological information measuring apparatus comprising the biological information measuring module according to claim 7.
